(19) United States
(12) Patent Application Publication
(10) Pub. No.: US 2024/0318162 A1
(43) Pub. Date: Sep. 26, 2024

(54) METHODS AND REAGENTS FOR ANALYZING NUCLEIC ACIDS

(71) Applicant: CUBIT DIAGNOSTICS, INC., Chicago, IL (US)

(72) Inventors: Samuel J. Hartman-Pickerill, Chicago, IL (US); Martine Dominique Lunke, North Saanich (CA); Matthew Dean McFarlane, Saanich (CA); Jackson William Price, Victoria (CA); Paul John Romaniuk, Victoria (CA)

(73) Assignee: CUBIT DIAGNOSTICS, INC., Chicago, IL (US)

(21) Appl. No.: 18/575,659

(22) PCT Filed: Jun. 30, 2022
(86) PCT No.: PCT/US22/35691

§ 371 Date: Dec. 29, 2023

Related U.S. Application Data
(60) Provisional application No. 63/217,507, filed on Jul. 1, 2021.
(60) Provisional application No. 63/217,521, filed on Jul. 1, 2021.

(51) Int. Cl. C12N 15/10; C12Q 1/6806

(52) U.S. Cl. CPC C12N 15/1006; C12Q 1/6806

(57) ABSTRACT

Methods of specifically capturing and processing nucleic acids from samples in a simplified, streamlined workflow. Media conditions and/or workflow improvements provided herein permit workflow steps such as cell lysis, nucleic acid denaturation, and target nucleic acid capture and immobilization to be performed in a “one-pot” reaction with minimal or no subsequent washing or rinsing steps prior to enzymatic processing. The media conditions include particular salt and/or detergent concentrations, and the workflow improvements include improved purification steps. The media conditions and workflow improvements effectively isolate target nucleic acid for downstream enzymatic processing reactions while avoiding levels of contaminants that inhibit such reactions.

### CROSS-REFERENCE TO RELATED APPLICATIONS

The present application is a national phase application under 35 U.S.C. 371 of International Application No. PCT/US2022/035691, filed Jun. 30, 2022, which claims priority to U.S. Application No. 63/217,507, filed Jul. 1, 2021, and U.S. Application No. 63/217,521, filed Jul. 1, 2021, the contents of all which are incorporated herein by reference in their entireties.

### SEQUENCE LISTING

The instant application contains a Sequence Listing which has been submitted in ASCII format and is hereby incorporated by reference in its entirety. The ASCII copy, created on Dec. 29, 2023, is named USPTO-231229-106398.006-Sequence_Listing.txt and is 19,963 bytes in size.

### FIELD OF THE INVENTION

The invention is directed to methods of specifically capturing and processing nucleic acids from samples in a simplified, streamlined workflow.

### BACKGROUND

The isolation and detection of specific nucleic acids in a sample can be used to determine the presence of particular biological entities in the sample (e.g., particular types of cells, bacteria, and/or viruses), detect the presence of mutations, or otherwise diagnose a disease state or detect a particular characteristic. Conventional workflows for isolating and detecting specific nucleic acids in samples with high sensitivity and specificity, however, employ a large number of separate, cumbersome processing steps and reagents that are highly inhibitory to downstream enzymatic reactions required for detection. Such steps may include lysing cells with detergents and chaotropic agents, non-specific binding of nucleic acids to a solid substrate, multiple washing steps utilizing alcohols and salts, and elution of said nucleic acid in a low salt buffer. These methods are effective in isolating nucleic acids but suffer from inhibition carry-over, for instance ethanol or salt carry-over, are not specific for the nucleic acid of interest, and are not well suited for automation within microfluidic or other consumable devices. Specific capture methods have been deployed that specifically hybridize the target nucleic acids with capture oligos. Unfortunately these methods also deploy chemistries that inhibit downstream enzymatic reactions, require multiple separate processing steps to wash away inhibitors and elute and amplify and/or detect the target nucleic acid with an enzymatic reaction, making them ill-suited for automation within microfluidic or other consumable devices. Separation of the aforementioned steps results from an incompatibility of conventional media and/or reaction conditions among the various steps. Streamlined workflows that eliminate or condense at least some of these steps while still providing high sensitivity and specificity would be useful for providing rapid, easy-to-use diagnostic tests, particularly within the microfluidic and consumable format. Streamlined workflows for isolating and processing specific nucleic acids in a sample are therefore needed.

### SUMMARY OF THE INVENTION

The invention provides methods for specifically capturing and processing nucleic acids from a sample in a simplified, streamlined workflow. An exemplary method is as follows. A clinical sample is first combined with a lysis/binding reagent (liquid buffer or dried reagents) that contain salt, buffer and either a detergent or proteinase K or both, and capture oligomers that are complementary to the target nucleic acid. Lysis and/or denaturation is performed using heat or pH, and hybridization is performed by lowering the temperature and/or pH such that the capture oligomers will hybridize to the specific target nucleic acid. The capture oligomer-target nucleic acid complex is then immobilized on a solid substrate, e.g. magnetic beads, membrane, frit, or other solid substrate. Liquid is removed, and the captured target nucleic acids are retained on the solid substrate. The resulting captured nucleic acids are substantially devoid of non-specific nucleic acids and concentrations of contaminants that would typically inhibit downstream enzymatic reactions. The captured nucleic acids are either washed or rinsed once with a salt-containing buffer or alternatively subjected directly to an enzymatic reaction (e.g., nucleic acid amplification, nucleic acid sequencing, CRISPR). Alternative exemplary workflows and/or processing steps are described elsewhere herein.

Media conditions and/or workflow improvements provided herein permit workflow steps such as cell lysis, nucleic acid denaturation, and target nucleic acid capture and immobilization to be performed in a “one-pot” reaction with few or no washing or rinsing steps. The media conditions include particular salt and/or detergent concentrations, and the workflow improvements include improved purification steps. The media conditions and workflow improvements effectively isolate target nucleic acid for downstream enzymatic processing reactions while avoiding levels of contaminants that inhibit such reactions.

An aspect of the invention is directed to methods of capturing and processing a target nucleic acid. The methods can comprise immobilizing a target nucleic acid on a solid substrate in contact with a first solution. The immobilizing can comprise a step of hybridizing the target nucleic acid to a capture oligomer configured to bind to the target nucleic acid to generate a target complex. The first solution can comprise water, salt, and, optionally, a first reagent comprising at least one of a detergent and a protease. The method can further comprise removing the first solution from the immobilized target complex, and then enzymatically processing the target nucleic acid.

In some versions, the first solution comprises the first reagent. In some versions, the first reagent comprises a detergent. In some versions, the detergent comprises an anionic detergent. In some versions, the detergent comprises dodecyl sulfate salt. In some versions, the dodecyl sulfate salt is present in the first solution in amount from 0.05% w/v to 3%, 3.5%, 4%, or 5% w/v. In some versions, the dodecyl sulfate salt comprises at least one of sodium dodecyl sulfate and lithium dodecyl sulfate. In some versions, the detergent comprises a lauroyl sarcosinate salt. In some versions, the detergent comprises sarkosyl. In some versions, the lauroyl sarcosinate salt is present in the first solution in an amount from 0.05% w/v to 3%, 3.5%, 4%, 4.5%, 5%, 5.5%, or 6% w/v.

In some versions, the first reagent comprises a protease. In some versions, the protease is present in the first solution in an amount of 3 to 300 Units. In some versions, the protease comprises proteinase K.

In some versions, the salt is present in the first solution in an amount effective to result in a molar ionic strength equivalent to 50 mM to 1 M NaCl.

In some versions, the methods comprise lysing cells and/or denaturing the target nucleic acid in the first solution.

In some versions, the lysing and/or denaturing comprises heating the first solution to a first temperature. In some versions, the first temperature is from 70° C. to 110° C. In some versions, the hybridizing comprises cooling the first solution to a second temperature. In some versions, the second temperature is from 30° C. to 75° C.

In some versions, the lysing and/or denaturing comprises increasing the pH of the first solution to a first pH. In some versions, the first pH is from pH 10 to pH 14. In some versions, the hybridizing comprises decreasing the pH of the first solution to a second pH. In some versions, the second pH is from pH 5 to pH 10.

In some versions, the immobilized target complex is not washed or is washed only once after the removing the first solution from the immobilized target complex and prior to the enzymatically processing the target nucleic acid. In some versions, the immobilized target complex is not rinsed or is rinsed three or fewer times, two or fewer times, or only once after the removing the first solution from the immobilized target complex and prior to the enzymatically processing the target nucleic acid. In some versions, the immobilized target complex is not washed after the removing the first solution from the immobilized target complex and prior to the enzymatically processing the target nucleic acid.

In some versions, the target nucleic acid is enzymatically processed as part of the immobilized target complex without eluting the target nucleic acid from the capture oligomer.

In some versions, the enzymatically processing the target nucleic acid comprises amplifying a target nucleic acid sequence comprised by the target nucleic acid.

In some versions, the solid substrate comprises a magnetic substrate, such as a magnetic bead, and removing the first solution comprises immobilizing the magnetic substrate with a magnetic field and separating the first solution from the immobilized magnetic bead.

In some versions, the solid substrate comprises filtering the first solution with the solid substrate through a porous substrate to separate the first solution from the solid substrate via size exclusion and thereby capture the solid substrate on or in the porous substrate. The solid substrate in such versions can comprise a bead, filament, etc. Some versions comprise enzymatically processing the target nucleic acid in the presence of the porous substrate. Some versions comprise enzymatically processing the target nucleic acid with the target nucleic acid immobilized on the solid substrate. Some versions further comprise, after the capturing the solid substrate on or in the porous substrate and prior to the enzymatic processing, contacting the porous substrate and the captured solid substrate with an enzymatic buffer. Some versions comprise enzymatically processing the target nucleic acid in the enzymatic buffer in the presence of the porous substrate. Some versions comprise enzymatically processing the target nucleic acid in the enzymatic buffer with the target nucleic acid immobilized on the solid substrate.

In some versions, the first solution comprises the first reagent; the first reagent comprises a detergent, wherein the detergent comprises dodecyl sulfate salt present in the first solution in amount from 0.05% w/v to 1%, 1.5%, 2%, 2.5%, or 3% w/v or a lauroyl sarcosinate salt present in the first solution in an amount from 0.05% w/v to 1%, 1.5%, 2%, 2.5%, 3%, 3.5%, or 4% w/v; the salt is present in the first solution in an amount effective to result in a molar ionic strength equivalent to 50 mM to 1 M NaCl; the method comprises lysing cells and/or denaturing the target nucleic acid in the first solution, wherein the lysing and/or denaturing comprises heating the first solution to a first temperature, wherein the first temperature is from 70° C. to 110° C.; the hybridizing comprises cooling the first solution from the first temperature to a second temperature, wherein the second temperature is from 30° C. to 75° C.; the immobilized target complex is not washed or is washed only once after the removing the first solution from the immobilized target complex and prior to the enzymatically processing the target nucleic acid; the target nucleic acid is enzymatically processed as part of the immobilized target complex without eluting the target nucleic acid from the capture oligomer; and the enzymatically processing the target nucleic acid comprises amplifying a target nucleic acid sequence comprised by the target nucleic acid. Optionally, the immobilized target complex is not washed after the removing the first solution from the immobilized target complex and prior to the enzymatically processing the target nucleic acid. Optionally, the method comprises binding the target complex to the solid substrate, the binding the target complex to the solid substrate comprises cooling the first solution to a temperature from 10° C. to 50° C., and the solid substrate is in contact with the first solution during the lysing and/or denaturing and also during the hybridizing. Optionally, the removing the first solution comprises filtering the first solution with the solid substrate through a porous substrate to separate the first solution from the bead via size exclusion and thereby capture the solid substrate on or in the porous substrate, and the method further comprises enzymatically processing the target nucleic acid with the target nucleic acid immobilized on the solid substrate and in the presence of the porous substrate.

Additional aspects of the invention are described elsewhere herein.

The objects and advantages of the invention will appear more fully from the following detailed description of the preferred embodiment of the invention made in conjunction with the accompanying drawings.

### BRIEF DESCRIPTION OF THE DRAWINGS

FIGS. 1A-1D show exemplary workflows of the invention. FIG. 1A. Full workflow with separate steps. FIG. 1B. Streamlined workflow with lysis, hybridization, and immobilization occurring in the same reaction solution. Beads can be captured using a magnet or on filter paper and target nucleic acid can be amplified directly off the beads or eluted before amplifying. FIG. 1C. Specific capture workflow on a lateral flow strip. FIG. 1D. Vertical flow capture using streptavidin and nitrocellulose.

FIG. 2. Time to amplification for LAMP reactions containing: A) 0 μl to 20 μl of eluates from specific capture samples processed with one wash step; B) 0 μl to 20 μl of eluates from specific capture samples processed with one rinse step; and C) 0 μl to 20 μl of eluates from specific capture samples processed without any wash or rinse step. The results are presented as average time to amplification in minutes and the 95% confidence interval is shown.

FIG. 3. Percent recovery of the SARS-COV-2 N-gene RNA after specific capture using lysis/hybridization buffers with various detergents.

FIG. 4. Percent recovery of intact DNA, sonicated DNA, RE-digested DNA and a 963-bp synthetic DNA fragment.

FIG. 5. Percent recovery of the nsp1 gene of SARS-COV-2 RNA or DNA with various denaturation temperatures.

FIG. 6. Effect of the distance between the qPCR primer and the capture oligomer binding sites on the % DNA recovery (A) and on the % DNA recovery relative to that obtained with the closest qPCR primer pair (B).

FIG. 7. Percent recovery of DNA with various template sizes.

FIG. 8A. Diagram of a filtration device.

FIG. 8B. Bead retention of the different membranes. The membrane after filtration is shown on the top, whereas the area of filter paper that was under the membrane during filtration is shown in the circles at the bottom.

FIGS. 9A-9D. Fluorescence data of the amplification curves for 50 μl RT-LAMP reactions with 7 mm membrane disks. The type of membrane and the pore size are indicated on each amplification plot.

FIGS. 10A and 10B. Fluorescence data of the real-time amplification of 100 μl RT-LAMP reactions with 11 mm membrane disks and Oligo-dT PMP. The type of membrane and the pore size are indicated on each amplification plot. The samples with the RNA added after the specific target capture are shown with round symbols, the samples with the RNA annealed to the beads are shown with square symbols.

FIG. 11. Lateral flow strip detection of SARS-COV-2 virus isolated using specific target capture with a size exclusion device and amplified by RT-LAMP.

FIG. 12. Lateral flow strip detection of inactivated Chlamydia trachomatis cells isolated using a size exclusion device and amplified using LAMP.

FIGS. 13A and 13B. Melting curves of the amplification products obtained by RT-LAMP with direct amplification on the magnetic particles (FIG. 13A) or with eluates (FIG. 13B).

### DETAILED DESCRIPTION OF THE INVENTION

An aspect of the invention is directed to methods of capturing and processing a target nucleic acid.

The target nucleic acid can be comprised by or derived from a sample. The sample can comprise a sample obtained or derived from a subject (i.e., a clinical sample), a synthetic sample, or any other type of sample potentially containing a target nucleic acid. Examples of clinical samples include whole blood, serum, plasma, sputum, saliva, nasopharyngeal swab, stool, anal swab, vaginal swab, urine, dry blood spot, penile swab, urethral swab, and skin swab. “Whole blood” as used herein refers to blood drawn from the body from which none of the components, such as plasma or platelets, has been removed. Whole blood can comprise components in addition to those originally present, such as EDTA and/or other components.

The target nucleic acid can include any type of nucleic acid. The target nucleic acid can comprise DNA or RNA. The nucleic acid can be single stranded or double stranded. Exemplary types of DNA include genomic DNA, cDNA, and extrachromosomal DNA, among others. Exemplary types of RNA include mRNA, tRNA, IRNA, and uRNA, among others. The target nucleic acid can comprise a sequence of interest. The sequence of interest, for example, can be a sequence indicative of, or unique to, a particular cell, pathogen, bacterium, virus, disease state, mutation status, genetic characteristic, or other item of interest.

The methods herein can comprise a number of steps performed in a first solution. The first solution preferably comprises water, salt, and, optionally, a first reagent. In some versions, the first solution is generated by combining a sample (such as a clinical sample) and one or more of water, salt and a first reagent.

The water included in the first solution can originate entirely from the sample (in which case there is no addition of additional water), can be added to the sample entirely from an external source (in which case there is no water originating from the sample itself), or a combination thereof. Accordingly, in some versions, the sample, prior to combining with the salt and/or first reagent, comprises all the water present in the first solution after the combining, and the combining comprises combining the sample to one or more of salt in dried form and the first reagent in dried form to generate the first solution without further addition of water. In some versions, the first solution is generated by combining water and, optionally, the salt and/or first reagent, with the sample.

The salt in the first solution can comprise any one or more monovalent salts, any one or more multivalent salts, or any combination thereof. Exemplary salts include calcium salts, copper salts, iron salts, selenium salts, potassium salts, magnesium salts, sodium salts, lithium salts, ammonium salts, nickel salts, tin salts, and zinc salts, among others. Suitable examples of such salts include CaCl₂, CuSO₄, FeSO₄, H₂SeO₃, KCl, KI, KH₂PO₄, MgCl₂, MgCO₃, MgSO₄, MnSO₄, Na₂HPO₄, Na₂SiO₃, NaCl, LiCl, NaH₂PO₄, NaHCO₃, NH₄VO₃, (NH₄)₆Mo₇O₂₄, NiCl₂, SnCl₂, ZnSO₄, and hydrates thereof.

The salt may be provided in the first solution at a concentration that provides a molar ionic strength equivalent to a molar ionic strength of a particular concentration of NaCl. In preferred versions, the salt is provided in the first solution at a concentration that provides a molar ionic strength equivalent to 0.001 mM to 2 M NaCl, such as a molar ionic strength equivalent to 0.001 mM to 1 M NaCl, such as a molar ionic strength equivalent to 0.001 mM, 0.01 mM, 0.05 mM, 0.1 mM, 0.5 mM, 1 mM, 25 mM, 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 500 mM, 600 mM, 700 mM, 800 mM, 900 mM, or 1 M NaCl or any range including and between any two of the foregoing values. Methods of determining ionic strength are well known in the art. See, e.g., Solomon, Theodros (2001). “The definition and unit of ionic strength.” Journal of Chemical Education. 78 (12): 1691. As outlined in the following examples, certain of these amounts of the salt in the first solution permit cell lysis, nucleic acid denaturation, capture oligomer-target nucleic acid hybridization, and target complex immobilization all to be performed in the first solution without altering the composition of the first solution or purifying select components from the first solution; enzymatically processing the target nucleic acid with no or minimal (such as only one) washing or rinsing of the immobilized target complex after removing the first solution therefrom; and no elution of the target nucleic acid from the immobilized target complex after removing the first solution therefrom.

The salt included in the first solution can originate entirely from the sample (in which case there is no addition of additional salt), can be added to the sample entirely from an external source (in which case there is no salt originating from the sample itself), or a combination thereof. Accordingly, in some versions, the sample, prior to combining with the water and/or first reagent, comprises all the salt present in the first solution after the combining, and the combining comprises combining the clinical sample with one or more of water and the first reagent to generate the first solution without further addition of salt. In some versions, the first solution is generated by combining salt and, optionally, water and/or the first reagent, with the sample.

The first reagent can comprise at least one of a detergent and a protease.

The detergent can be included as a first reagent in the first solution to assist in the lysis of cells present in the sample, among other functions. Exemplary detergents for including in the first solution as a first reagent include anionic detergents, cationic detergents, nonionic detergents, and zwitterionic detergents. Anionic detergents are preferred. Exemplary anionic detergents include soaps, alkylbenzene sulfonates, alkyl sulfonates, alkyl sulfonates, alkyl sulfates, salts of fluorinated fatty acids, silicones, fatty alcohol sulfates, polyoxyethylene fatty alcohol ether sulfates, α-olefin sulfonate, polyoxyethylene fatty alcohol phosphates ether, alkyl alcohol amide, alkyl sulfonic acid acetamide, alkyl succinate sulfonate salts, amino alcohol alkylbenzene sulfonates, naphthenates, alkylphenol sulfonate and polyoxyethylene monolaurate. Specific exemplary anionic detergents include sodium octyl sufate, potassium oleate, sodium dodecyl sulfate, lithium dodecyl sulfate, butylnaphthalenesulfonic acid sodium salt, sodium decyl sulfate, sodium 1-butanesulfonate, sodium dodecylbenzenesulphonate, sodiuim stearate, magnesium stearate, 1-dodecanesulfonic acid sodium salt, sodium allylsulfonate, dodecylbenzenesulfonic acid sodium salt, calcium dodecylbenzene sulfonate, ammonium lauryl sulfate, and sodium lauryl polyoxyethylene ether sulfate, among others. Preferred detergents include dodecyl sulfate salts such as sodium dodecyl sulfate (SDS) or lithium dodecyl sulfate, or others. Other preferred detergents include lauroyl sarcosinate salts, such as sarkosyl (sodium lauroyl sarcosinate).

The detergent is preferably included in the first solution in an amount of 0.01% to 20% w/v, such as 0.05% to 6% w/v, 0.05% to 5% w/v, 0.05% to 4% w/v, or 0.05% to 3% w/v. Exemplary amounts include 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, or 6.0% w/v or any range including and between any two of the foregoing values. Preferred ranges for dodecyl sulfate salts include 0.05% to 5% w/v, 0.05% to 4% w/v, or 0.05% to 3% w/v. Preferred ranges for lauroyl sarcosinate salts include 0.05% to 6% w/v. As outlined in the following examples, certain of these amounts of the detergent in the first solution permit cell lysis, nucleic acid denaturation, capture oligomer-target nucleic acid hybridization, and target complex immobilization all to be performed in the first solution without altering the composition of the first solution or purifying select components from the first solution; enzymatically processing the target nucleic acid with no or minimal (such as only one) washing or rinsing of the immobilized target complex after removing the first solution therefrom; and no elution of the target nucleic acid from the immobilized target complex after removing the first solution therefrom.

The protease can be included as a first reagent in the first solution to digest proteins such as nucleases or other proteins present in the sample. The proteins may be released into solution after cell lysis. The digestion of proteins such as nucleases can protect the nucleic acids in the sample from nuclease attack. Any protease or combination of proteases can be included as a first reagent in the first solution. A preferred protease is proteinase K, which is a broad-spectrum protease. The protease is preferably included in the first solution an amount of 1-600 Units (U), for example 3 to 300 Units. Exemplary amounts include 3 U, 6 U, 9 U, 12 U, 15 U, 18 U, 21 U, 24 U, 27 U, 30 U, 40 U, 50 U, 60 U, 70 U, 80 U, 90 U, 100 U, 150 U, 200 U, 250 U, or 300 U or any range including and between any two of the foregoing values. Definitions of units for specific proteases are known in the art. A unit of proteinase K is defined as an amount of proteinase K that hydrolyzes urea-denatured hemoglobin to produce color equivalent to 1.0 μmole of tyrosine per min at pH 7.5 at 37° C. (color by Folin-Ciocalteu reagent). As outlined in the following examples, certain of these amounts of the protease in the first solution permit cell lysis, nucleic acid denaturation, capture oligomer-target nucleic acid hybridization, and target complex immobilization all to be performed in the first solution without altering the composition of the first solution or purifying select components from the first solution; enzymatically processing the target nucleic acid with no or minimal (such as only one) washing or rinsing of the immobilized target complex after removing the first solution therefrom; and no elution of the target nucleic acid from the immobilized target complex after removing the first solution therefrom.

Some versions of the invention comprise lysing cells and/or denaturing the target nucleic acid in the first solution.

In some versions, the lysing and/or denaturing comprises incubating the first solution at a first temperature for a time. The first temperature is preferably from 70° C. to 110° C., such as 70, 75, 80, 85, 90, 95, 100, 105, or 110° C., or any range including and between any two of the foregoing values. The time is preferably from 30 seconds or less to 10 minutes or more, such as 30 seconds, 1 minute, 2 minutes, 3 minutes, 4 minutes, 5 minutes, 6 minutes, 7 minutes, 8 minutes, 9 minutes, 10 minutes, 15 minutes, or 20 minutes, or any range including and between any two of the foregoing values.

In some versions, the lysing and/or denaturing comprises adjusting the pH of the first solution to a first pH. The first pH is preferably a pH from pH 10 to pH 14, such as pH 10, pH 10.5, pH 11, pH 11.5, pH 12, pH 12.5, pH 13, pH 13.5, or pH 14, or any range including and between any two of the foregoing values.

Some versions of the invention comprise hybridizing the target nucleic acid to a capture oligomer in the first solution to thereby generate a target complex. The capture oligomer is a oligomer configured to bind to the target nucleic acid. The capture oligomer May comprise a sequence that is sufficiently complementary to a corresponding sequence on the target nucleic acid to permit the hybridization of the capture oligomer to the target nucleic acid in the first solution at a given temperature. In some versions, the given temperature is from 30° C. to 75° C. such as 30, 35, 40, 45, 50, 55, 60, 65, 70, or 75° C. or any range including and between any two of the foregoing values.

In some versions, the hybridizing comprises incubating the first solution at a second temperature for a time. The second temperature is preferably from 30 to 75° C., such as 30, 35, 40, 45, 50, 55, 60, 65, 70, or 75° C., or any range including and between any two of the foregoing values. The time is preferably from 30 seconds or less to 10 minutes or more, such as 30 seconds, 1 minute, 2 minutes, 3 minutes, 4 minutes, 5 minutes, 6 minutes, 7 minutes, 8 minutes, 9 minutes, 10 minutes, 15 minutes, or 20 minutes, or any range including and between any two of the foregoing values. If the lysing and/or denaturing comprises incubating the first solution at the first temperature, the hybridizing can comprise cooling the first solution to the second temperature.

In some versions, the hybridizing comprises adjusting the pH of the first solution to a second pH. The first pH is preferably a pH from pH 5 to pH 10, such as pH 5, pH 5.5, pH 6, pH 6.5, pH 7, pH 7.5, pH 8, pH 8.5, pH 9, pH 9.5, or pH 10, or any range including and between any two of the foregoing values. If the lysing and/or denaturing comprised adjusting the pH of the first solution to the first pH, the hybridizing can comprise decreasing the pH of the first solution to the second pH.

In some versions, the capture oligomer hybridizes to a position on the target nucleic acid in close proximity to a target nucleic acid sequence. As used herein, “target nucleic acid sequence” refers to sequence on a target nucleic acid that is enzymatically processed (e.g., reverse transcribed, copied, cleaved, etc.) in additional to any additional sequence required for such enzymatic processing (e.g., a restriction enzyme recognition sequence, CRISPR recognition sequence, etc.). In some versions, the capture oligomer hybridizes to a position on the target nucleic acid no more than 10,000 bp away from the target nucleic acid sequence, such as no more than no more than 9500 bp, no more than 9000 bp, no more than 8500 bp, no more than 8000 bp, no more than 7500 bp, no more than 7000 bp, no more than 6500 bp, no more than 6000 bp, no more than 5500 bp, no more than 5000 bp, no more than 4500 bp, no more than 4000 bp, no more than 3500 bp, no more than 3000 bp, no more than 2500 bp, no 20) more than 2000 bp, no more than 1750 bp, no more than 1500 bp, no more than 1250 bp, no more than 1000 bp, no more than 900 bp, no more than 950 bp, no more than 900 bp, no more than 850 bp, no more than 800 bp, no more than 750 bp, no more than 700 bp, no more than 650 bp, no more than 600 bp, no more than 550 bp, no more than 500 bp, no more than 450 bp, no more than 400 bp, no more than 350 bp, no more than 300 bp, no more than 250 bp, no more than 200 bp, no more than 150 bp, no more than 100 bp, or no more than 25 bp away from the target nucleic acid sequence. Such distances are counted from the base on the target nucleic acid to which the capture oligomer hybridizes that is most proximate to the target nucleic acid sequence to the base on the target nucleic acid sequence most proximate to the binding site of the capture oligomer. In other words, the distances outlined above constitute the number of bases on the target nucleic acid between the capture oligomer binding site and the target sequence. In cases in which the enzymatic processing of the target nucleic acid sequence involves amplification, the target nucleic sequence is defined as the bases on the target nucleic acid that are amplified.

Some versions of the invention comprise immobilizing the target nucleic acid on a solid substrate in contact with the first solution. The solid substrate can comprise a bead, a membrane, or any other type of solid substrate. The solid substrate should be capable of maintaining the target nucleic acid in a solid phase when in contact with the liquid phase of the first solution and when the liquid phase of the first solution is removed from the solid phase. Exemplary beads include magnetic beads (e.g., Dynabeads® (ThermoFisher Scientific), polymeric beads (e.g., polystyrene), glass beads, etc. Exemplary membranes include polymer membranes (e.g., polyethersulfone, nylon, polytetrafluoroethylene, polycarbonate, nitrocellulose), glass fiber membranes, cellulose membranes, and highly matrixed membranes.

The format in which the target nucleic acid is immobilized on the solid substrate depends on whether the capture oligomer is pre-bound to the solid substrate prior to hybridizing to the target nucleic acid or is configured to bind to the solid substrate after or during hybridizing to the target nucleic acid. In versions in which the capture oligomer is pre-bound to the solid substrate prior to hybridizing to the target nucleic acid, the immobilization of the target nucleic acid on the solid substrate occurs with the hybridization of the capture oligomer to the target nucleic acid. In versions in which the capture oligomer is configured to bind to the solid substrate after or during hybridizing to the target nucleic acid, the immobilization of the target nucleic acid on the solid substrate comprises a step of binding a target complex comprising the target nucleic acid and the capture oligomer either directly or indirectly to the solid substrate via the capture oligomer.

Regardless of whether the capture oligomer is pre-bound to the solid substrate prior to hybridizing to the target nucleic acid or is configured to bind to the solid substrate after or during hybridizing to the target nucleic acid, the binding of the oligomer to the solid substrate can be mediated by a specific binding pair. “Specific binding pair” refers to a pair of binding moieties (e.g., a “first binding moiety” and a “second binding moiety”) that are capable of specifically binding to each other. The capture oligomer, for example, can comprise a first binding moiety of the specific binding pair, the first binding moiety can be bound to or be capable of binding to a second binding moiety of the specific binding pair, and the second binding moiety of the specific binding pair can be bound to or be capable of binding to the solid substrate. Various exemplary specific binding pairs include streptavidin and biotin, hybridizable nucleic acid sequences (e.g., a poly(A) sequence and a poly(T) sequence), an antibody and an antigen of the antibody, a G-quadruplex structure and a G-quadruplex-binding protein; an aptamer and an aptamer target, and an ion/anion binding pair. Other specific binding pairs suitable for the purposes herein are known in the art.

In some versions, the specific binding pair are specific binding pairs that bind to each other at a specific temperature or temperature range. Hybridizable nucleic acid sequences, such as poly(A) and poly(T) sequences for example, can be configured to bind to each other at a particular temperature or temperature range. In some versions of the invention, the specific binding pairs are configured to bind to each other at a temperature of 10 to 50° C., such as 10, 15, 20, 25, 30, 35, 40, 45, or 50° C. or any range including and between any two of the foregoing values. Exemplary hybridizable nucleic acid sequences that bind to each other at such temperatures include poly dA and poly dT sequences each having a chain length of 10 to 50 bases, such as: 10, 15, 20, 25, 30, 35, 40, 45, or 50 bases or any range including and between any two of the foregoing values. Other hydridizable nucleic acid sequences can be designed to bind to each other at such temperatures. Accordingly, in versions of the invention employing specific binding pairs that bind to each other such temperatures, immobilizing the target nucleic acid on the solid substrate can comprise incubating the first solution at a third temperature for a time. The third temperature is preferably from 10 to 50° C., such as 10, 15, 20, 25, 30, 35, 40, 45, or 50° C. or any range including and between any two of the foregoing values. The time is preferably from 30 seconds or less to 10 minutes or more, such as 30 seconds, 1 minute, 2 minutes, 3 minutes, 4 minutes, 5 minutes, 6 minutes, 7 minutes, 8 minutes, 9 minutes, 10 minutes, 15 minutes, or 20 minutes, or any range including and between any two of the foregoing values. The immobilizing the target nucleic acid on the solid substrate can comprise cooling the first solution to the third temperature, such as from the second 20) temperature to the third temperature or from the first temperature to the second temperature and ultimately to the third temperature. In some versions of the invention, first solution can be heated to a temperature of 70° C. to 110° C. prior to the immobilizing to generate a heated solution whereby lysis and/or nucleic acid denaturation can occur. The heated solution can then be exposed to a temperature of 20-25° C. (e.g., room temperature) for a time sufficient to cool the first solution with the target nucleic acid therein to a temperature of 65° C. or lower to thereby hybridize the target nucleic acid to the capture oligomer and then a temperature from 10 to 50° C. for immobilization. Mere passage of the first solution through the second and third temperatures during cooling can be effective for hybridization and immobilization.

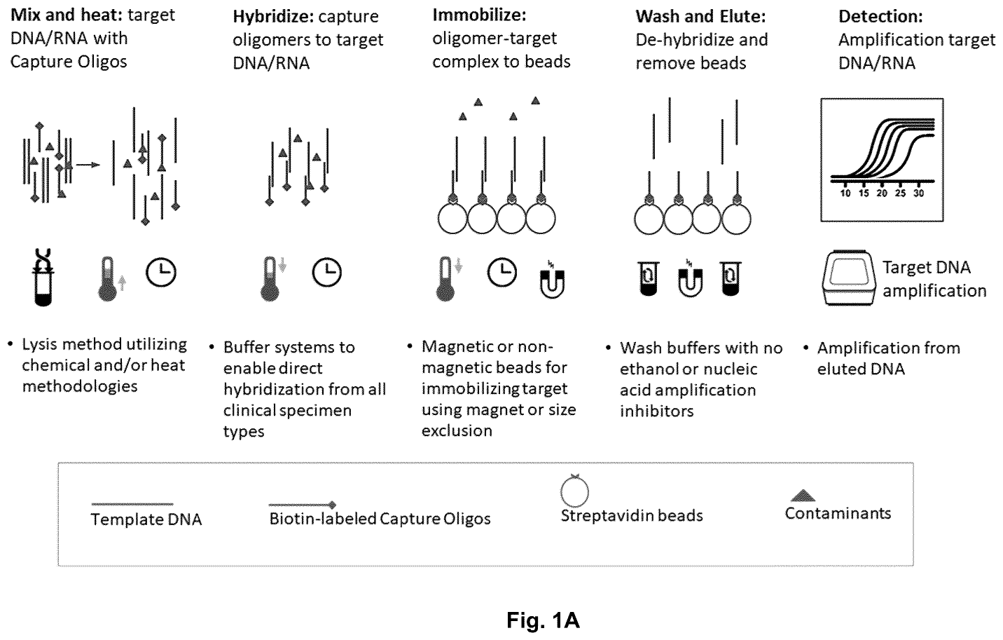

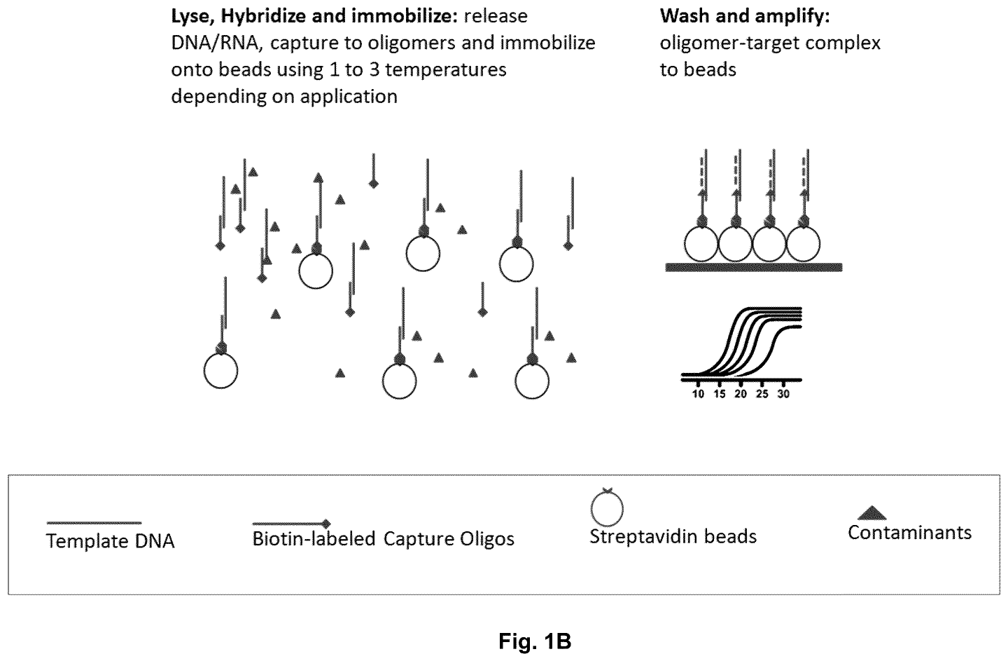

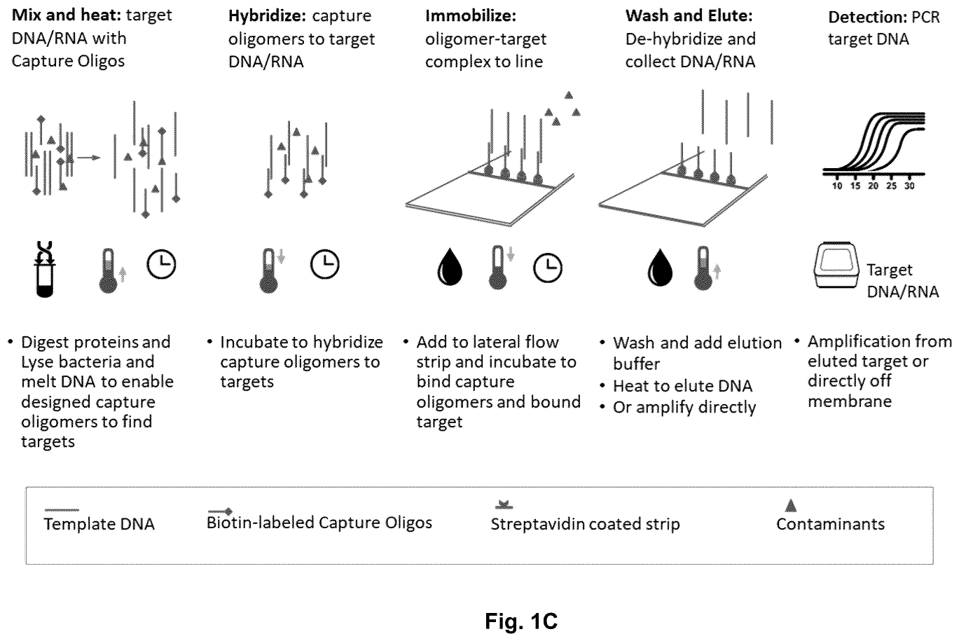

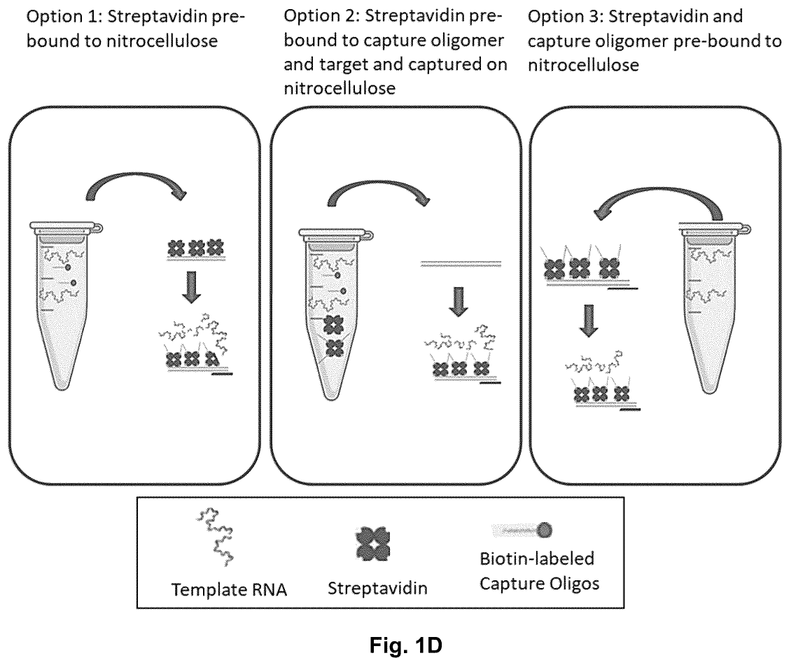

In some versions, the solid substrate is in contact with the first solution during the hybridizing and, optionally, the lysing and/or denaturing in a “one-pot” method for lysing and/or denaturing, hybridizing, and immobilizing. In some versions, the second binding moiety is bound to the solid substrate during the hybridizing and, optionally, the lysing and/or denaturing. See FIG. 1B, and Exemplary Methods 1 and 2 below for examples of workflows encompassing such aspects.

In some versions, the solid substrate is first contacted with the first solution after the hybridizing. The second binding moiety can be bound to the solid substrate during the first contacting. See FIG. 1C, Option 1 of FIG. 1D, and Exemplary Methods 3 and 8 examples of workflows encompassing such aspects. The solid substrate can comprise a porous substrate in such versions, and the first contacting can comprises flowing the first solution through the porous substrate. The porous substrate can comprise a first region and a second region. The first region lacks the second binding moiety bound thereto and second region comprises the second binding moiety bound thereto. The flowing can comprise flowing the first solution through first region prior to flowing the first solution through the second region. The porous substrate can comprise a lateral flow strip or a vertical flow sandwich. See FIG. 1C and Exemplary Method 3 for examples of workflows encompassing such aspects.

In some versions, the immobilizing can comprise binding the second binding moiety to the solid substrate. Prior to binding the second binding moiety to the solid substrate, the target nucleic acid can be hybridized to the capture oligomer, and the first binding moiety on the capture oligomer can be bound to the second binding moiety. The second binding moiety can comprise a protein, such as streptavidin or any other protein. The solid substrate can comprise a non-specific protein-binding substrate. The non-specific protein-binding substrate can comprise at least one of nitrocellulose, nylon, and polyvinylidene difluoride (PVDF). See Option 2 of FIG. 1D and Exemplary Method 9 for examples of workflows encompassing such aspects. In some versions, the solid substrate comprises a moiety that specifically binds the second binding moiety. See Exemplary Method 9 for examples of workflows encompassing such aspects.

In some versions, the first binding moiety is bound to the second binding moiety and the second binding moiety is bound to the solid substrate prior to the hybridizing. See Option 3 of FIG. 1D for an example of a workflow encompassing such an aspect.

After generating the immobilized target complex, the methods of the invention can comprise removing the first solution from the immobilized target complex.

In some versions, the solid substrate comprises a magnetic substrate, such as a magnetic bead, and removing the first solution comprises immobilizing the magnetic substrate with a magnetic field and separating the first solution from the immobilized magnetic substrate. See FIG. 1B and Exemplary Method 1 (among others) for examples of workflows encompassing such an aspect.

In some versions, removing the first solution comprises filtering the first solution with the solid substrate through a porous substrate to separate the first solution from the solid substrate via size exclusion and thereby capture the solid substrate on or in the porous substrate. The solid substrate in such versions can comprise a bead, a filament, etc. “Porous substrate” refers to any porous solid or semi-solid substrate that permits a fluid such as a liquid to flow therethrough. The porous substrate may be configured to permit certain solids or particles having a certain size or physicochemical characteristic to flow therethrough, while capturing or filtering others. Examples include polymeric, ceramic, or other types of filters or frits or any of the membranes described herein. The porous substrate encompasses the “solid support” described in the Exemplary Methods outlined below. The porous substrate can have any pore size suitable for the methods described herein. Exemplary pore sizes include 0.01 μm, 0.02 μm, 0.03 μm, 0.04 μm, 0.05 μm, 0.06 μm, 0.07 μm, 0.08 μm, 0.09 μm, 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, or 10 μm or any range including and between any two of the foregoing values. Pore sizes above and below the foregoing values are also acceptable, provided the pore size is suitable for capturing the solid substrate. In some versions, the pore size is equal to or less than the size (diameter for beads) of the solid substrate. In some versions, the pore size is less than the size (diameter for beads) of the solid substrate.) In some versions, the filtering comprises filtering the first solution with the solid substrate through the porous substrate via capillary action, gravity, a pressure gradient or a combination thereof. In some versions, the porous substrate with the captured solid substrate is contacted with an enzymatic buffer after capturing the solid substrate on or in the porous substrate and prior to enzymatic processing the target nucleic acid. The enzymatic buffer can comprise a nucleic acid amplification buffer. In some versions, the target nucleic acid in the enzymatic buffer is enzymatically processed in the presence of the porous substrate. In some versions, the enzymatic processing comprises nucleic acid amplification. In some versions, the target nucleic acid is enzymatically processed with the target nucleic acid immobilized on the solid substrate. In other words, the target nucleic acid is not eluted from the capture oligomer prior to the enzymatic processing, such as nucleic acid amplification wherein a target nucleic acid sequence comprised by the target nucleic acid is amplified directly from the target nucleic acid while immobilized on the solid substrate. See FIG. 1B and Exemplary Method 2 (among others) for examples of workflows encompassing such aspects.

After removing the first solution from the immobilized target complex, the methods of the invention can comprise enzymatically processing the target nucleic acid. “Enzymatically processing” refers to any method of processing a nucleic acid that involves an enzyme. “Processing” in this context refers to the involvement in any way (e.g., as a substrate, reactant, etc.) of the nucleic acid in an enzymatic reaction. Examples of enzymatic processing include nucleic acid amplification with nucleic acid polymerases, nucleic acid sequencing, restriction enzyme digestion, CRISPR-Cas processing, such as with Cas12a or Cas13a (see, e.g., Wang M, Zhang R, Li J. CRISPR/cas systems redefine nucleic acid detection: Principles and methods. Biosens Bioelectron. 2020 Oct. 1; 165:112430), among others. As used herein, “nucleic acid amplification” encompasses reverse transcription of RNA to DNA, copying of DNA, and combinations thereof. The nucleic acid amplification can comprise any method suitable for amplifying nucleic acids. Exemplary methods comprise thermocycling amplification, such as the polymerase chain reaction (PCR), and isothermal amplification. A number of isothermal amplification methods are known in the art. These include transcription mediated amplification (TMA), nucleic acid sequence-based amplification (NASBA), signal mediated amplification of RNA technology (SMART), strand displacement amplification (SDA), nicking enzyme amplification reaction (NEAR), rolling circle amplification (RCA), loop-mediated isothermal amplification of DNA (LAMP), isothermal multiple displacement amplification (MDA), helicase-dependent amplification (HDA), single primer isothermal amplification (SPIA), and cross primed amplification (CPA). See, e.g., Notomi et al. 2000 (Notomi T, Okayama H, Masubuchi H, Yonekawa T, Watanabe K, Amino N, Hase T. Loop-mediated isothermal amplification of DNA. Nucleic Acids Res. 2000 Jun. 15; 28(12):E63), U.S. Pat. Nos. 6,410,278; 6,743,605; 6,764,821; 7,494,790; 7,468,245; 7,485,417; 7,713,691; 8,133,989; 8,206,902; 8,288,092; 8,445,664; 8,486,633; and 8,906,621. Software and other methods for designing primers suitable for use in such isothermal amplification methods are well-known in the art. See, e.g., PrimerExplorer LAMP primer designing software from Eiken Chemical, Kimura et al. 2011 (Kimura Y, de Hoon M J, Aoki S, Ishizu Y, Kawai Y, Kogo Y, Daub C O, Lezhava A, Arner E, Hayashizaki Y. Optimization of turn-back primers in isothermal amplification. Nucleic Acids Res. 2011 May; 39(9): e59), and others.

By virtue of the media conditions (e.g., first reagent concentration(s)) and/or workflow improvements (e.g., filtering the immobilized target complex through a porous substrate) in some embodiments of the invention, the immobilized target complex in some versions of the invention is not washed or rinsed or is minimally washed or rinsed after removing the first solution from the immobilized target complex and prior to enzymatically processing the target nucleic acid. In some versions, the immobilized target complex is not washed or is washed three or fewer times, two or fewer times, or only once after removing the first solution from the immobilized target complex and prior to enzymatically processing the target nucleic acid. In some versions, the immobilized target complex is not washed or is washed only once after removing the first solution from the immobilized target complex and prior to enzymatically processing the target nucleic acid. In some versions, the immobilized target complex is not washed after removing the first solution from the immobilized target complex and prior to enzymatically processing the target nucleic acid. In some versions, the immobilized target complex is washed only once after removing the first solution from the immobilized target complex and prior to enzymatically processing the target nucleic acid. In some versions, the immobilized target complex is not rinsed or is rinsed three or fewer times, two or fewer times, or only once after removing the first solution from the immobilized target complex and prior to enzymatically processing the target nucleic acid. In some versions, the immobilized target complex is not rinsed or is rinsed only once after removing the first solution from the immobilized target complex and prior to enzymatically processing the target nucleic acid. In some versions, the immobilized target complex is not rinsed after removing the first solution from the immobilized target complex and prior to enzymatically processing the target nucleic acid. In some versions, the immobilized target complex is rinsed only once after removing the first solution from the immobilized target complex and prior to enzymatically processing the target nucleic acid. In some versions, the immobilized target complex is not washed or rinsed or is washed and/or rinsed three or fewer times, two or fewer times, or only once after removing the first solution from the immobilized target complex and prior to enzymatically processing the target nucleic acid. In some versions, the immobilized target complex is not washed or rinsed or is washed and/or rinsed only once after removing the first solution from the immobilized target complex and prior to enzymatically processing the target nucleic acid. In some versions, the immobilized target complex is not washed or rinsed after removing the first solution from the immobilized target complex and prior to enzymatically processing the target nucleic acid. In some versions, the immobilized target complex is washed and/or rinsed only once after removing the first solution from the immobilized target complex and prior to enzymatically processing the target nucleic acid.

“Wash” as used herein refers to the suspension of the immobilized target complex in a wash solution followed by removal of the wash solution from the immobilized target complex. For example, washing occurs with magnetic beads when they are suspended in a wash solution and then subsequently magnetically pulled against a container surface while the wash solution is removed.

“Rinse” as used herein refers to the mere contacting of the immobilized target complex in a wash solution without suspending the immobilized target complex therein, followed by removal of the wash solution from the immobilized target complex. Rinsing without washing can occur with magnetic beads if the magnetic beads are merely contacted with a wash solution while magnetically pulled against a container surface, without releasing the magnetic beads from the container surface in suspension. Rinsing without washing can also occur when the solid substrate in the immobilized target complex is a membrane and a wash solution is flushed over or through the membrane. Rinsing without washing can also occur when the solid substrate in the immobilized target complex is a bead that is captured in or on a porous substrate and a wash solution is flushed over or through the porous substrate. In such cases, the immobilized target complex is not suspended in the wash solution and is merely contacted with it.

In some versions, the target nucleic acid is enzymatically processed as part of the immobilized target complex without eluting the target nucleic acid from the capture oligomer. In other words, the target nucleic acid is not eluted from the capture oligomer in the immobilized target complex prior to enzymatically processing the target nucleic acid.

In some versions, a precursor nucleic acid is fragmented prior to the immobilizing to thereby generate the target nucleic acid. The fragmenting can be performed using any method that digests nucleic acids into smaller nucleic acids. Exemplary methods include DNase treatment, high pH treatment, agitation, enzymatic cleavage (e.g., restriction enzyme cleavage), and sonication, among others. The high pH treatment can involve adjusting pH to a value of pH 10 to pH 14, such as pH 10, pH 10.5, pH 11, pH 11.5, pH 12, pH 12.5, pH 13, pH 13.5, or pH 14 or any range including and between any two of the foregoing values. The pH treatment can occur with or without heat, for example, from 50 to 110° C., such as 50, 55, 60, 65, 70, 75, 80, 85, 90, 95, 100, 105, or 110° C. or any range including and between any two of the foregoing values. Agitation may also be applied and in combination with a pH treatment, heat treatment, or both. The fragmenting is preferably sufficient to generate a target nucleic acid having a size less than 5000 bp, less than 4750 bp, less than 4500 bp, less than 4250 bp, less than 4000 bp, less than 3750 bp, less than 3500 bp, less than 3250 bp, less than 3000 bp, less than 2750 bp, less than 2500 bp, less than 2250 bp, less than 2000 bp, less than 1750 bp, less than 1500 bp, less than 1250 bp, or less than 1000 bp. Fragmentation may have the added benefit of allowing unwanted long-chain nucleic acid to pass through a porous substrate which in an un-fragmented state may clog the substrate limiting processing of a sample.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXEMPLARY METHODS

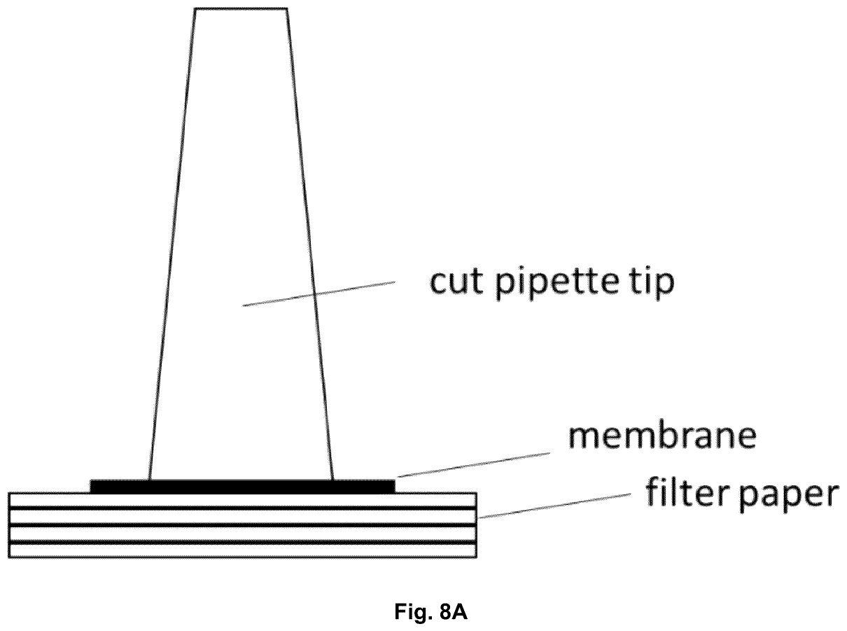

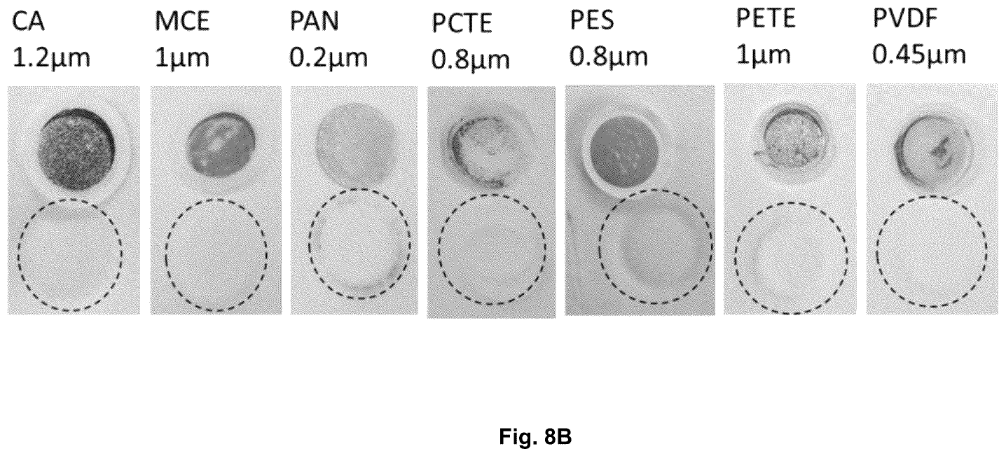

1. Direct from bead, zero or one-wash protocol:
   a. A clinical sample (e.g., whole blood, serum, plasma, sputum, saliva, nasopharyngeal swab, stool, anal swab, vaginal swab, urine, dry blood spot, penile swab, urethral swab, and skin swab, etc., or any other clinical sample described herein or known in the art) is added and mixed into a lysis/binding buffer to create a solution with the following composition:
      i. Sodium dodecyl sulfate (SDS) or other dodecyl sulfate salt (e.g., lithium dodecyl sulfate): 0.01%-20% w/v, for example 0.05% to 5% w/v, such as: 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% w/v or any range including and between any two of the foregoing values.
      ii. Salt: Any monovalent salt, multivalent salt, or combination thereof providing a molar ionic strength equivalent to 0.001 mM to 2 M NaCl, such as a molar ionic strength equivalent to 0.001 mM to 1 M NaCl or 50 mM to 1 M NaCl, such as a molar ionic strength equivalent to 0.001 mM, 0.01 mM, 0.05 mM, 0.1 mM, 0.5 mM, 1 mM, 25 mM, 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 500 mM, 600 mM, 700 mM, 800 mM, 900 mM, or 1 M NaCl or any range including and between any two of the foregoing values.
      iii. Optionally, proteinase K to improve processing: 1-600 Units, for example 3 to 300 Units such as: 3 U, 6 U, 9 U, 12 U, 15 U, 18 U, 21 U, 24 U, 27 U, 30 U, 40 U, 50 U, 60 U, 70 U, 80 U, 90 U, 100 U, 150 U, 200 U, 250 U, or 300 U or any range including and between any two of the foregoing values.
      iv. Capture oligomers complementary to the target(s) of interest and labeled with a binding moiety, for example, poly dA of a chain length of 10 to 50 bases, such as: 10, 15, 20, 25, 30, 35, 40, 45, or 50 bases or any range including and between any two of the foregoing values.
      v. Magnetic beads with a binding moiety complementary to the capture oligomers, for example poly dT of a chain length of 10 to 50 bases, such as: 10, 15, 20, 25, 30, 35, 40, 45, or 50 bases or any range including and between any two of the foregoing values.
   b. The mixture is heated to a temperature that denatures the dsDNA and also assists in the lysis of any pathogens, for example, 70 to 110° C., such as 70, 75, 80, 85, 90, 95, 100, 105, or 110° C. or any range including and between any two of the foregoing values.
      i. Alternatively, pH may be used to denature the dsDNA by increasing the pH, for example to a value between pH 10 to pH 14, such as pH 10, pH 10.5, pH 11, pH 11.5, pH 12, pH 12.5, pH 13, pH 13.5, or pH 14 or any range including and between any two of the foregoing values.
   c. The mixture is brought down to a temperature that enables the hybridization of the capture oligomers to a DNA target, for example 30 to 75° C., such as 30, 35, 40, 45, 50, 55, 60, 65, 70, or 75° C. or any range including and between any two of the foregoing values.
      i. Alternatively, if pH was used for denaturation, the pH can be reduced, for example to a value between pH 5 and pH 10, such as pH 5, pH 5.5, pH 6, pH 6.5, pH 7, pH 7.5, pH 8, pH 8.5, pH 9, pH 9.5, or pH 10 or any range including and between any two of the foregoing values.
   d. The temperature is reduced to a temperature at which to allow the poly dA of the capture oligomers and the poly dT of the magnetic beads to hybridize, for example 10 to 50° C., such as 10, 15, 20, 25, 30, 35, 40, 45, or 50° C. or any range including and between any two of the foregoing values.
   e. The beads are collected on the side of the tube wall using the application of a magnetic field.
   f. The mixture is removed, leaving just the beads which contain the capture oligomers and target hybridized complex.
   g. The magnetic field is removed.
   h. Optionally, a wash buffer is added to further remove inhibitors. The wash buffer should contain sufficient salts (monovalent, multivalent, or combination thereof) to maintain hybridization of the target to the capture oligomers, For example, 50 mM to 500 mM of a monovalent salt such as NaCl or equivalent thereto, such as an equivalent to: 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, or 500 mM NaCl or any range including and between any two of the foregoing values.
   i. The beads are collected on the side of the tube wall using the application of a magnetic field.
   j. The mixture is removed, leaving just the beads which contain the capture oligomers and target hybridized complex.
   k. The magnetic field is removed.
   l. Amplification buffer is added directly to the beads and incubated to encourage a nucleic acid amplification reaction (e.g. polymerase chain reaction (PCR), loop-mediated isothermal amplification (LAMP), etc.).
2. Size-exclusion, filter-based capture:
   a. The protocol according to above is used up until step 1d.
   b. The bead sample mixture is then passed through a filter, frit or other solid support with pore sizes that will trap the beads and allow the sample mixture to flow through.
      i. Preferably the filter is such that filtration occurs passively via capillary action through the use of a blotter pad, for example Whatman gel blotting paper.
      ii. Alternatively, positive or negative pressure may be applied.
      iii. For example, for use with a 1 μm bead size with <1 μm pore size can be used. Other filters and beads may be used so long as the filter pore size is such that it captures the beads by size exclusion. (See example set up in FIG. 8A):
         1) An example membrane successfully used is the PES-08 membrane, a polyethersulfone membrane with a pore size of 0.8 μm, from Sterlitech.
         2) Alternatively, cellulose acetate membranes may be used.
         3) In some instances, for example for RNA templates, the PETE-1.0 membranes—a polyester track etch membrane with a pore size of 1 μm, also from Sterlitech—may be used.
         4) In other instances, a highly matrixed membrane, for example, the Whatman Fusion 5 membrane, may be used.
   c. The solid support containing beads is then added to a tube with an amplification buffer or an amplification buffer is added to the solid support containing beads and the nucleic acid is amplified using standard methods.
3. Lateral/vertical flow-based capture with a capture oligomer binding region:
   a. A clinical sample (e.g., whole blood, serum, plasma, sputum, saliva, nasopharyngeal swab, stool, anal swab, vaginal swab, urine, dry blood spot, penile swab, urethral swab, and skin swab, etc., or any other clinical sample described herein or known in the art) is added and mixed into a lysis/binding buffer to create a solution with the following composition:
      i. Sodium dodecyl sulfate (SDS) or other dodecyl sulfate salt (e.g., lithium dodecyl sulfate): 0.01%-20% w/v, for example 0.05% to 5% w/v, such as: 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% w/v or any range including and between any two of the foregoing values.
      ii. Salt: Any monovalent salt, multivalent salt, or combination thereof providing a molar ionic strength equivalent to 0.001 mM to 2 M NaCl, such as a molar ionic strength equivalent to 0.001 mM to 1 M NaCl, such as a molar ionic strength equivalent to 0.001 mM, 0.01 mM, 0.05 mM, 0.1 mM, 0.5 mM, 1 mM, 25 mM, 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 500 mM, 600 mM, 700 mM, 800 mM, 900 mM, or 1 M NaCl or any range including and between any two of the foregoing values.
      iii. Optionally, proteinase K to improve processing: 1-600 Units, for example 3 to 300 Units such as: 3 U, 6 U, 9 U, 12 U, 15 U, 18 U, 21 U, 24 U, 27 U, 30 U, 40 U, 50 U, 60 U, 70 U, 80 U, 90 U, 100 U, 150 U, 200 U, 250 U, or 300 U or any range including and between any two of the foregoing values.
      iv. Capture oligomers complementary to the target(s) of interest and labeled with a binding moiety, for example, biotin.
         1) Alternatively, the binding moiety is poly dA of a chain length of 10 to 50 bases, such as: 10, 15, 20, 25, 30, 35, 40, 45, or 50 bases or any range including and between any two of the foregoing values.
   b. The mixture is heated to a temperature that denatures the dsDNA and also assists in the lysis of any pathogens, for example, 70 to 110° C., such as 70, 75, 80, 85, 90, 95, 100, 105, or 110° C. or any range including and between any two of the foregoing values.
      i. Alternatively, pH may be used to denature the dsDNA by increasing the pH, for example to a value between pH 10 to pH 14, such as pH 10, pH 10.5, pH 11, pH 11.5, pH 12, pH 12.5, pH 13, pH 13.5, or pH 14 or any range including and between any two of the foregoing values.
   c. The mixture is brought down to a temperature that enables the hybridization of the capture oligomers to a DNA target, for example 30 to 75° C., such as 30, 35, 40, 45, 50, 55, 60, 65, 70, or 75° C. or any range including and between any two of the foregoing values.
      i. Alternatively, if pH was used for denaturation, the pH can be reduced, for example to a value between pH 5 and pH 10, such as pH 5, pH 5.5, pH 6, pH 6.5, pH 7, pH 7.5, pH 8, pH 8.5, pH 9, pH 9.5, or pH 10 or any range including and between any two of the foregoing values.
   d. The sample mixture is added to membrane, filter, particles, beads or solid support that contains an oligomer binding region.
      i. For example, the binding region may be a region coated with streptavidin.
      ii. Alternatively, for example the binding region may be a region coated with poly dT of a chain length of 10 to 50 bases, such as: 10, 15, 20, 25, 30, 35, 40, 45, or 50 bases or any range including and between any two of the foregoing values.
      iii. For example, the solid support may be a lateral flow strip or a vertical flow sandwich. The material of said solid support may be any material that enables the attachment of an oligomer binding region. For example, the material may be nitrocellulose. In another example, the material may be a multimeric polymer. In another example the material may be a highly matrixed membrane, for example the WHATMAN Fusion 5 membrane (Whatman plc, Little Chalfont, Buckinghamshire, United Kingdom).
      iv. Alternatively, the sample mixture may be combined with a bead that contains an oligomer binding region, for example streptavidin, that is then added to a membrane, filter or solid support with pore sizes that will trap the beads and allow the sample mixture to flow through, similar to Exemplary Method 2 above. The beads may be immobilized and localized utilizing other means, such as magnetic force in the case where the beads are para-magnetic.
   e. The sample mixture is allowed to flow past or through the capture oligomer binding region to a water chamber or pad leaving the capture oligomers and hybridized target.
   f. The oligomer binding region is then added to a tube with an amplification buffer and the nucleic acid is amplified using standard methods.
4. To improve recovery from genomic DNA or long DNA:
   a. A method to fragment the DNA can be utilized. Exemplary methods include:
      i. Short DNase treatment.
      ii. High pH, for example to a value between pH 10 to pH 14, such as pH 10, pH 10.5, pH 11, pH 11.5, pH 12, pH 12.5, pH 13, pH 13.5, or pH 14 or any range including and between any two of the foregoing values. With or without heat, for example from 50 to 110° C., such as 50, 55, 60, 65, 70, 75, 80, 85, 90, 95, 100, 105, or 110° C. or any range including and between any two of the foregoing values. With or without agitation.
      iii. Enzymatic cleavage, e.g. Nco1 restriction enzyme.
      iv. Sonication.
   b. An alternative to fragmentation is utilizing divalent salts, for example MgCl₂ at a concentration of 5 mM to 1 M, for example 10 mM to 100 mM free Mg²⁺, such as 10, 15, 20, 25, 30, 35, 40, 45, 50, 60, 70, 80, 90, or 100 mM or any range including and between any two of the foregoing values.
5. To reduce volume and further improve recovery and ease of processing:
   a. Concentrate buffers to <100% of input volume, for example 10 to 50% v/v of the sample volume, such as 10, 15, 20, 25, 30, 35, 40, 45, or 50% v/v or any range including and between any two of the foregoing values.
   b. Optionally buffers may be dried down to reduce volume to a minimum and/or enable ease of processing. For example, the salts and detergents may be present dry on a filter or solid support and be reconstituted by the sample and/or addition of liquid.
6. Methods to improve a nucleic acid amplification reaction or other enzymatic reaction, e.g. nucleic acid amplification, with a nucleotide following the specific capture of a target nucleic acid:
   a. Capture oligomers, specific to the target can be designed so that the 3′ end can engage in the amplification reaction by being extended by a polymerase. For example, one or more capture oligomers can be designed to also be a primer in a downstream amplification reaction. The capture oligomers may preferably be designed so that they are the outer primers in an amplification reaction so that additional inside primers can engage in further amplification following extension of the capture oligomer.
   b. To prevent non-specific amplification and/or non-specific formation of an enzyme-nucleic acid complex, which would reduce efficiency of the target specific reaction:
      i. The capture oligomer binding moiety can be a moiety that is not capable of engaging in the amplification reaction. For instance, a non-nucleic acid binding moiety. For instance, streptavidin/biotin.
      ii. In the case where the poly dA and poly dT binding moiety or some other nucleic acid based binding moiety is preferred, as in Exemplary Method 1, the following approaches can be used:
         1) One or more inverted nucleotides (e.g., an inverted dT) or L-DNA nucleotides (enantiomers of native nucleotides), can be added to the 3′ end of the binding region. This prevents an enzyme, e.g. a polymerase, from forming a complex with the nucleotide. Alternative molecules that prevent interaction with an enzyme, for instance a C18 spacer may be used to “cap” the 3′ end of the binding region.
         2) The orientation of the nucleic acid based binding moiety may be reversed so that only the 5′ region is exposed and therefore cannot form a complex with the polymerase.
7. Methods to capture multiple targets on unique solid substrates:
   Capture oligomers specific to a target may be labeled with a unique binding moiety that is different from the binding moiety of a separate binding moiety used for a different target. Any number of binding moieties may be selected and paired with a solid substrate that has complementary binding moieties. For instance, target A can have capture oligomers that are labeled with biotin and are captured on a solid substrate labeled with streptavidin. Target B can have capture oligomers labeled with fluorescein isothiocyanate (FITC) and are captured on a solid substrate labeled with anti-FITC. Target C can utilize Digoxigenin (DIG) and anti-DIG, and so-on. This enables the controlled capture and localization of any number of targets. For instance, multiple targets could be localized on different regions of a membrane or frit. This may be combined with localized enzymatic reactions, e.g. an amplification reaction, enabling highly multiplexed reactions of targets localized by the unique binding moieties chosen for each target.
8. Direct bead protocol with zero or one-wash protocol using biotin/streptavidin beads with separate addition of beads:
   a. A clinical sample (e.g., whole blood, serum, plasma, sputum, saliva, nasopharyngeal swab, stool, anal swab, vaginal swab, urine, dry blood spot, penile swab, urethral swab, and skin swab, etc., or any other clinical sample described herein or known in the art) is added and mixed into a lysis/binding buffer to create a solution with the following composition:
      i. Sodium dodecyl sulfate (SDS) or other dodecyl sulfate salt (e.g., lithium dodecyl sulfate): 0.01%-20% w/v, for example 0.05% to 5% w/v, such as: 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% w/v or any range including and between any two of the foregoing values.
      ii. Salt: Any monovalent salt, multivalent salt, or combination thereof providing a molar ionic strength equivalent to 0.001 mM to 2 M NaCl, such as a molar ionic strength equivalent to 0.001 mM to 1 M NaCl, such as a molar ionic strength equivalent to 0.001 mM, 0.01 mM, 0.05 mM, 0.1 mM, 0.5 mM, 1 mM, 25 mM, 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 500 mM, 600 mM, 700 mM, 800 mM, 900 mM, or 1 M NaCl or any range including and between any two of the foregoing values.
      iii. Optionally, proteinase K to improve processing: 1-600 Units, for example 3 to 300 Units such as: 3 U, 6 U, 9 U, 12 U, 15 U, 18 U, 21 U, 24 U, 27 U, 30 U, 40 U, 50 U, 60 U, 70 U, 80 U, 90 U, 100 U, 150 U, 200 U, 250 U, or 300 U or any range including and between any two of the foregoing values.
      iv. Capture oligomers complementary to the target(s) of interest and labeled with a binding moiety, for example, biotin.
         1) Alternatively, the binding moiety is poly dA of a chain length of 10 to 50 bases, such as: 10, 15, 20, 25, 30, 35, 40, 45, or 50 bases or any range including and between any two of the foregoing values.
   b. The mixture is heated to a temperature that denatures the dsDNA and also assists in the lysis of any pathogens, for example, 70 to 110° C., such as 70, 75, 80, 85, 90, 95, 100, 105, or 110° C. or any range including and between any two of the foregoing values.
      i. Alternatively, pH may be used to denature the dsDNA by increasing the pH, for example to a value between pH 10 to pH 14, such as pH 10, pH 10.5, pH 11, pH 11.5, pH 12, pH 12.5, pH 13, pH 13.5, or pH 14 or any range including and between any two of the foregoing values.
c. The mixture is brought down to a temperature that enables the hybridization of the capture oligomers to a DNA target, for example 30 to 75° C., such as 30, 35, 40, 45, 50, 55, 60, 65, 70, or 75° C. or any range including and between any two of the foregoing values.
   i. Alternatively, if pH was used for denaturation, the pH can be reduced, for example to a value between pH 5 and pH 10, such as pH 5, pH 5.5, pH 6, pH 6.5, pH 7, pH 7.5, pH 8, pH 8.5, pH 9, pH 9.5, or pH 10 or any range including and between any two of the foregoing values.
   d. Magnetic beads with a binding moiety capable of binding the capture oligomers are added. For example, Streptavidin coated beads.
      i. Alternatively, for example the binding region may be a region coated with poly dT of a chain length of 10 to 50 bases, such as: 10, 15, 20, 25, 30, 35, 40, 45, or 50 bases or any range including and between any two of the foregoing values.
   e. The beads are collected on the side of the tube wall using the application of a magnetic field.
   f. The mixture is removed, leaving just the beads which contain the capture oligomers and target hybridized complex.
   g. The magnetic field is removed.
   h. Optionally, a wash buffer is added to further remove inhibitors.
   i. The beads are collected on the side of the tube wall using the application of a magnetic field.
   j. The mixture is removed, leaving just the beads which contain the capture oligomers and target hybridized complex.
   k. The magnetic field is removed.
   l. Amplification buffer is added directly to the beads and incubated to encourage a nucleic acid amplification reaction (e.g. PCR, LAMP etc.).
9. Capture oligomers pre-bound to streptavidin and captured by a protein binding region:
   a. A clinical sample (e.g., whole blood, serum, plasma, sputum, saliva, nasopharyngeal swab, stool, anal swab, vaginal swab, urine, dry blood spot, penile swab, urethral swab, and skin swab, etc., or any other clinical sample described herein or known in the art) is added and mixed into a lysis/binding buffer to create a solution with the following composition:
      i. Sodium dodecyl sulfate (SDS) or other dodecyl sulfate salt (e.g., lithium dodecyl sulfate): 0.01%-20% w/v, for example 0.05% to 5% w/v, such as: 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% w/v or any range including and between any two of the foregoing values.
      ii. Salt: Any monovalent salt, multivalent salt, or combination thereof providing a molar ionic strength equivalent to 0.001 mM to 2 M NaCl, such as a molar ionic strength equivalent to 0.001 mM to 1 M NaCl, such as a molar ionic strength equivalent to 0.001 mM, 0.01 mM, 0.05 mM, 0.1 mM, 0.5 mM, 1 mM, 25 mM, 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 500 mM, 600 mM, 700 mM, 800 mM, 900 mM, or 1 M NaCl or any range including and between any two of the foregoing values.
      iii. Optionally, proteinase K to improve processing: 1-600 Units, for example 3 to 300 Units such as: 3 U, 6 U, 9 U, 12 U, 15 U, 18 U, 21 U, 24 U, 27 U, 30 U, 40 U, 50 U, 60 U, 70 U, 80 U, 90 U, 100 U, 150 U, 200 U, 250 U, or 300 U or any range including and between any two of the foregoing values.
      iv. Capture oligomers complementary to the target(s) of interest and labeled with a binding moiety, preferably biotin.
      v. Free Streptavidin.
   b. The mixture is heated to a temperature that denatures the dsDNA and also assists in the lysis of any pathogens, for example, 70 to 110° C., such as 70, 75, 80, 85, 90, 95, 100, 105, or 110° C. or any range including and between any two of the foregoing values.
      i. Alternatively, pH may be used to denature the dsDNA by increasing the pH, for example to a value between pH 10 to pH 14, such as pH 10, pH 10.5, pH 11, pH 11.5, pH 12, pH 12.5, pH 13, pH 13.5, or pH 14 or any range including and between any two of the foregoing values.
   c. The mixture is brought down to a temperature that enables the hybridization of the capture oligomers to a DNA target, for example 30 to 75° C., such as 30, 35, 40, 45, 50, 55, 60, 65, 70, or 75° C. or any range including and between any two of the foregoing values.
      i. Alternatively, if pH was used for denaturation, the pH can be reduced, for example to a value between pH 5 and pH 10, such as pH 5, pH 5.5, pH 6, pH 6.5, pH 7, pH 7.5, pH 8, pH 8.5, pH 9, pH 9.5, or pH 10 or any range including and between any two of the foregoing values.
   d. The sample mixture is added to membrane, filter or solid support that is composed of a material that binds proteins and/or specifically streptavidin.
      i. For example, the filter is composed of nitrocellulose, nylon or another non-specific protein-binding material.
      ii. For example, the solid support is a lateral flow strip or a vertical flow sandwich.
   e. The sample mixture is allowed to flow past or through the capture oligomer binding region to a water chamber or pad leaving the capture oligomers and hybridized target.
   f. The membrane, filter or solid support is then added to a tube with an amplification buffer and the nucleic acid is amplified using standard methods, or an amplification buffer is added to the membrane, filter or solid support and the nucleic acid is amplified using standard methods.
10. For the biotin-streptavidin method described above in Exemplary Method 8, capture oligomers labeled with two biotin molecules can be used to improve the biotin-streptavidin interaction:
11. Alternative detergents:
   a. Preferably anionic detergents. For example, SDS and Sarkosyl.
12. Lysis/hybridization buffer may contain EDTA: 0.000001-4 M, for example 0.000001-1 M, such as: 0.000001 mM, 0.00001 mM, 0.0001 mM, 0.001 mM, 0.01 mM, 0.1 mM, 1 mM, 25 mM, 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 500 mM, 600 mM, 700 mM, 800 mM, 900 mM, or 1 M or any range including and between any two of the foregoing values.
13. Salt and detergent combination:
   a. If dodecyl sulfate salt is 0.05% to 1% w/v then lithium and sodium as dominant salt in the “first solution” both work well. If higher than 1% w/v dodecyl sulfate salt, lithium greatly reduces precipitation compared to sodium (precipitation is much worse with presence of potassium salts). This is important in some sample types. For example, whole blood generally requires a higher range of dodecyl sulfate to maintain sample processability in a process with quick heating, and may require, or be improved, with lithium as the minor or dominant salt.
14. Lower salt may be preferred to improve the speed of bead collection, for example a molar ionic strength equivalent to 0.001 mM to 200 mM NaCl such as an equivalent to: 0 mM, 25 mM, 50 mM, 75 mM, 100 mM, 125 mM, 150 mM, 175 mM, or 200 mM NaCl or any range including and between any two of the foregoing values.
15. ssDNA/RNA:
   a. No denaturation required.
16. Capture oligomer should preferably be designed proximate to the target region. Preferably <10,000 bp's from the target region, more preferably <1000 bp's from the target region.
17. Lateral flow method requiring no heat:
   a. A clinical sample (e.g., whole blood, serum, plasma, sputum, saliva, nasopharyngeal swab, stool, anal swab, vaginal swab, urine, dry blood spot, penile swab, urethral swab, and skin swab, etc., or any other clinical sample described herein or known in the art) is added and mixed into a lysis/binding buffer to create a solution with the following composition:
      i. Sodium dodecyl sulfate (SDS) or other dodecyl sulfate salt (e.g., lithium dodecyl sulfate): 0.01%-20% w/v, for example 0.05% to 5% w/v, such as: 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% w/v or any range including and between any two of the foregoing values.
      ii. Salt: Any monovalent salt, multivalent salt, or combination thereof providing a molar ionic strength equivalent to 0.001 mM to 2 M NaCl, such as a molar ionic strength equivalent to 0.001 mM to 1 M NaCl, such as a molar ionic strength equivalent to 0.001 mM, 0.01 mM, 0.05 mM, 0.1 mM, 0.5 mM, 1 mM, 25 mM, 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 500 mM, 600 mM, 700 mM, 800 mM, 900 mM, or 1 M NaCl or any range including and between any two of the foregoing values.
      iii. Sufficient alkaline material to denature dsDNA for example to a value between pH 10 to pH 14, such as pH 10, pH 10.5, pH 11, pH 11.5, pH 12, pH 12.5, pH 13, pH 13.5, or pH 14 or any range including and between any two of the foregoing values.
      iv. Optionally, proteinase K to improve processing: 1-600 Units, for example 3 to 300 Units such as: 3 U, 6 U, 9 U, 12 U, 15 U, 18 U, 21 U, 24 U, 27 U, 30 U, 40 U, 50 U, 60 U, 70 U, 80 U, 90 U, 100 U, 150 U, 200 U, 250 U, or 300 U or any range including and between any two of the foregoing values.
      v. Capture oligomers complementary to the target(s) of interest and labeled with a binding moiety, for example, biotin.
         1) Alternatively, the binding moiety is poly dA of a chain length of 10 to 50 bases, such as: 10, 15, 20, 25, 30, 35, 40, 45, or 50 bases or any range including and between any two of the foregoing values.
   b. Add sufficient acid to reduce pH and allow for renaturation of the DNA and hybridization. The pH can be reduced, for example to a value between 5 and 10, such as pH 5, pH 5.5, pH 6, pH 6.5, pH 7, pH 7.5, pH 8, pH 8.5, pH 9, pH 9.5, or pH 10 or any range including and between any two of the foregoing values.
   c. The sample mixture is made to flow over a filter or solid support that contains an oligomer binding region.
      i. For example, the binding region is a region coated with streptavidin.
      ii. Alternatively, for example the binding region may be a region coated with poly dT of a chain length of 10 to 50 bases, such as: 10, 15, 20, 25, 30, 35, 40, 45, or 50 bases or any range including and between any two of the foregoing values.
      iii. Preferably the solid support is a lateral flow strip or a vertical flow sandwich.
   d. The sample mixture is allowed to flow past or through the capture oligomer binding region to a water chamber or pad leaving the capture oligomers and hybridized target.
   e. The oligomer binding region is then added to a tube with an amplification buffer and the nucleic acid is amplified using standard methods, or an amplification buffer is added to the binding region and is amplified using standard methods.
18. The methods described herein for purifying and amplifying nucleic acids may also or alternatively be utilized with suitable modifications to purify, amplify and detect other molecules of interest, including proteins, lipids, carbohydrate, cells and cellular structures, exosomes, metal ion and organometallic compounds. Such methods may be combined with target and/or signal amplification processes, such as self-propagating signal amplification processes. These include, for example, a metal-organic supramolecular system, such as the use of a supramolecular allosteric catalyst that catalyzes an exponential increase in an acetate ion through an acyl transfer reaction. See Yoon H J, Mirkin C A. PCR-like cascade reactions in the context of an allosteric enzyme mimic. J Am Chem Soc. 2008 Sep. 3; 130(35):11590-1. Also see Sun X, Shabat D, Phillips S T, Anslyn E V. Self-Propagating Amplification Reactions for Molecular Detection and Signal Amplification: Advantages, Pitfalls, and Challenges. J Phys Org Chem. 2018 Aug.; 31(8):e3827. The methods may be simply combined with the self-propagating signal amplification process to improve signal to noise. Alternatively, the methods may catalyze the amplification process by purifying and/or presenting an active reagent that “triggers” a target and/or signal amplification reaction.

### EXAMPLES

### Materials Used in the Examples

Hybridization (Hyb) buffer or Lysis/Hybridization (Lys/Hyb) buffer: Tris buffer containing a detergent and optionally salt to enable hybridization of the capture oligomer to the target of interest and optionally denaturation and lysis.
Capture oligomer (oligo): Nucleic acid that is complementary to the target of interest and includes a binding moiety (e.g. biotin or polyA).
PMP: paramagnetic particles.
Streptavidin beads or Streptavidin PMP: paramagnetic particles with covalently linked streptavidin, such as the Dynabeads MyOne Streptavidin C1 beads (MyOneC1 beads).
Oligo-dT beads or Oligo-dT PMP: paramagnetic particles with poly dT covalently linked on the surface for binding with the polyA portion of specific capture oligomers, such as the Sera-Mag Oligo (dT)₁₄ PMPs from Cytiva, in which the DNA oligonucleotide 5′-TTTTTTTTTTTTTT-3′ (SEQ ID NO:96) is covalently attached to the bead.
Wash buffer: Tris buffer containing salt.
Elution buffer (EB): low salt Tris buffer.

### Example 1

This example illustrates a typical workflow for specific target capture from a biological sample using streptavidin beads. The biological sample can be but is not limited to a nasal swab, a vaginal swab, blood or urine. Alternatively, the sample can be a simulated nasal matrix, a simulated vaginal fluid or blood certified as pathogen-free in which an artificial DNA or RNA template is added. 100 μl of the sample is mixed with 500 μl of a lysis/hybridization buffer comprising 30 mM Tris buffer pH 7.5, 225 mM NaCl, 3.6% SDS and 30 mM EDTA. 8-16 pmoles of target-specific, biotin-labelled capture oligomers are added and the mixture is heated for a denaturation step of 10 minutes at 95° C., followed by a hybridization step of 20 minutes at 60° C. 200 μg of washed Dynabeads MyOne Streptavidin C1 beads (Invitrogen) are added to the sample, which is then incubated at room temperature for 10 minutes with end-to-end rotation for the immobilization step. The sample is placed in a magnetic field and the streptavidin beads are allowed to collect to one side of the container. The liquid is removed and the container is removed from the magnetic field. The beads are re-suspended with either 100 μl or 600 μl of a first wash buffer comprising 50 mM Tris pH7.5, 150 mM NaCl, 1 mM EDTA and 0.1% SDS and the container is placed back in the magnetic field until the beads collect to the side of the container. The wash buffer is removed and a second wash step is then performed with either 100 μl or 600 μl of a second wash buffer comprising 10 mM Tris buffer pH 7.5 and 0.01% Tween-20 using the same method described above. The PMP are then re-suspended in 20 μl of an elution buffer comprising 10 mM Tris buffer pH 7.5 and 1 mM EDTA. The sample is heated for 3 minutes at 75° C. and placed back in the magnetic field to collect the PMP to one side of the container. The liquid fraction containing the eluted sample is collected and used for downstream applications.

### Example 2

This example illustrates a typical workflow for specific target capture from a biological sample using Oligo-dT PMP. The biological sample can be but is not limited to a nasal swab, a vaginal swab, urine or blood. Alternatively, the sample can be a simulated nasal matrix, a simulated vaginal fluid or blood certified as pathogen-free in which an artificial DNA or RNA template is added. 100 μl of the sample is mixed with 504 μl of a lysis/hybridization buffer comprising 120 mM Tris buffer pH 7.5, 12 mM EDTA, 48 mM ammonium sulfate, 120 mM lithium chloride, 0.12% SDS, 100 μg PMP and 8 to 16 pmoles of target-specific capture probes with a poly(A) moiety at the 3′ end. The sample is mixed by inverting the container and incubated for 10 minutes at 95° C. for the denaturation step, then 10 minutes at 60° C. for the hybridization step and then 10 minutes at room temperature for the immobilization step. The sample is placed in a magnetic field and the magnetic beads are allowed to collect to one side of the container. The liquid is removed and the beads are washed 0, 1 or 2 times with 600 μl of a wash buffer comprising 10 mM Tris pH 7.5, 150 mM sodium chloride, 1 mM EDTA and 0.01% Tween-20, using the method described in Example 1. The PMP are then re-suspended in 20 μl of an elution buffer comprising 10 mM Tris buffer pH 7.5. The sample is heated for 2 minutes at 80° C. and placed back in the magnetic field to collect the PMP to one side of the container. The liquid fraction containing the eluted sample is collected and used for downstream applications.

### Example 3

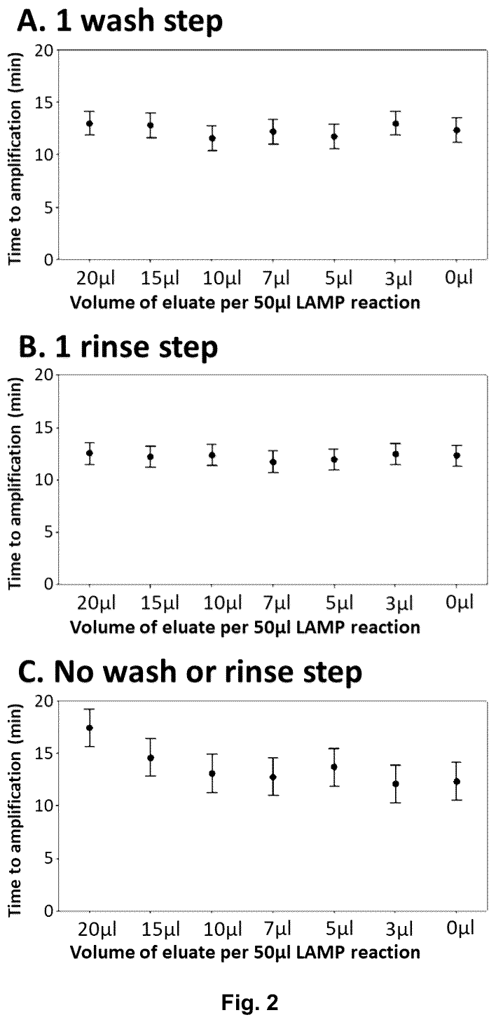

This example uses the specific capture method with Oligo-dT PMP and shows that the steps in which the beads are washed with the wash buffer can be simplified without causing any inhibition of the downstream LAMP amplification reaction. Here the steps were simplified to one quick rinse of the PMPs collected to the side of the container, or alternatively wash and rinse steps were eliminated altogether with only a minimal effect on the downstream amplification reaction. The simplification or elimination of the wash step is beneficial for the adaptation of the protocol to a simple cartridge for home use. The samples comprised a simulated vaginal fluid with the following composition: 60 mM NaCl, 25 mM KOH, 3 mM Ca(OH)₂, 0.27 μM bovine serum albumin, 22.2 mM lactic acid, 16.7 mM acetic acid, 1.7 mM glycerol, 6.7 mM urea, 27.8 mM glucose and 1.5% mucin, pH 4.2. Thirty samples were processed as described in Example 2 up to the beginning of the wash steps. The captured oligomers used are listed in Table 1. The samples were placed in the magnetic field. The first samples were rinsed once with the wash buffer using the wash buffer and protocol described in Example 2. The next 10 samples were placed in the magnetic field until the PMP had collected to the side of the container. The lysis/hybridization buffer was removed. The wash buffer was added and then removed without removing the container from the magnetic field so that the PMP remained in a tight pellet on the side of the container (“rinse”). For the final samples the wash and any rinse step was eliminated altogether. The samples were then eluted with 20 μl elution buffer as described in Example 2 and the 10 samples for each treatment group were pooled together. The eluates were then tested for their inhibitory effect on a LAMP reaction. For each treatment group, either 0 μl, 3 μl, 5 μl, 7 μl, 10 μl, 15 μl or 20 μl of the eluate pool was added to a 50 μl LAMP reaction. The template (500c of an artificial dsDNA fragment containing the pGB8-D gene of the cryptic plasmid of Chlamydia trachomatis) was added to each LAMP reaction. Each LAMP reaction contained 1× iB4 buffer (Optigene, UK), 3 mM magnesium sulfate, 0.4 mM each dNTP, 1 μM Syto-9, 1 M Betaine, 16 U GspF and 1× LAMP primers. The LAMP primer sequences are listed in Table 1. The reactions were incubated for 30 minutes at 65° C. in a CFX96 Touch Real-Time PCR Detection System thermocycler (Bio-Rad) with a read every 30 seconds. The results show that there is no inhibition of the LAMP reaction when up to 20 μl of the eluate from the samples with either 1 wash step or 1 rinse step is used in a 50 μl LAMP reaction. There is a slight inhibition of the LAMP reaction when 20 μl of the eluate from the samples without any wash or rinse step is used in a 50 μl LAMP reaction, but no significant inhibition when 15 μl or less of the same eluate is used (FIG. 2 and Table 2).

TABLE 1
DNA Capture oligomers and LAMP primers used in Example 3

| Capture oligomers | Sequence | Amount |
|---|---|---|
| CT_CP-cap840R | CTTCGAGCAACCGCTGTGACGGAGTACAAAAA | 2 pmoles |
| | AAAAAAAAAAAAAAAAAAAAAAAAA (SEQ ID | |
| | NO: 1) | |
| CT_CP-cap881R | CTCAGACTCCGACATAATGAAGGTCACAAAAA | 2 pmoles |
| | AAAAAAAAAAAAAAAAAAAAAAAAA (SEQ ID | |
| | NO: 2) | |
| CT_CP-cap560F | CCTCCTTGGAGCATTGTCTGGGCGATCAAAAAA | 2 pmoles |
| | AAAAAAAAAAAAAAAAAAAAAAAA (SEQ ID | |
| | NO: 3) | |
| CT_CP-cap505F | GCTCTTTTAGGAAGGATGCTGTTTGCAAAAAAA | 2 pmoles |
| | AAAAAAAAAAAAAAAAAAAAAAA (SEQ ID | |
| | NO: 4) | |
| NG_ISNgo2-328R | ACCGGCAATACTGCGCTATCAGTTTTGCAAAAA | 4 pmoles |
| | AAAAAAAAAAAAAAAAAAAAAAAAA (SEQ ID | |
| | NO: 5) | |
| NG_ISNgo2-613F | TGACCGCAGCCGTATTGTTTCATCATCAAAAAA | 4 pmoles |
| | AAAAAAAAAAAAAAAAAAAAAAAA (SEQ ID | |
| | NO: 6) | |
| | | Final |
| | | concentration |
| LAMP primers | Sequence | (μM) |
| CT-LAMP4-F3 | CTGCTTGAGAGAACGTGCG (SEQ ID NO: 7) | 0.2 |
| CT-LAMP4-B3 | CTC TGA AGT CTT AAG CTT GGA GAT | 0.2 |
| | (SEQ ID NO: 8) | |
| CT-LAMP4-FIP | TCGGTCAACGAAGAGGTTTTGCGATTTGC | 1.4 |
| | CTTAACCCCACC (SEQ ID NO: 9) | |
| CT-LAMP4-BIP | AATCCTCTTTTCTGTCTGACGGTCAGATTT | 1.4 |
| | GTTTCCAACAAGCTACC (SEQ ID NO: 10) | |
| CT-LAMP4-LF | TTT TGT CTT CGT AAC TCG CT (SEQ ID | 0.7 |
| | NO: 11) | |
| CT-LAMP4-LB | TCT TAA GCT GGG AGA AAG AAA T (SEQ | 0.7 |
| | ID NO: 12) | |

TABLE 2
Inhibition of LAMP reactions by eluates from specific capture
samples with 1 wash, 1 rinse or no washes/rinses.

| | No Washes/Rinses | | | 1 Wash | | | 1 Rinse | | |
|---|---|---|---|---|---|---|---|---|---|
| | TTA | | # pos- | TTA | | # pos- | TTA | | # pos- |
| Input | (min) | SD | itives | (min) | SD | itives | (min) | SD | itives |
| 20 μl | 17.4 | 2.9 | 3/3 | 13 | 0.5 | 3/3 | 12.6 | 0.4 | 3/3 |
| Eluate | | | | | | | | | |
| 15 μl | 14.6 | 1.2 | 3/3 | 12.8 | 1.1 | 3/3 | 12.2 | 1.1 | 3/3 |
| Eluate | | | | | | | | | |
| 10 μl | 13.1 | 1.7 | 3/3 | 11.6 | 0.7 | 3/3 | 12.4 | 0.3 | 3/3 |
| Eluate | | | | | | | | | |
| 7 μl | 12.8 | 0.6 | 3/3 | 12.2 | 1.4 | 3/3 | 11.7 | 1.3 | 3/3 |
| Eluate | | | | | | | | | |
| 5 μl | 13.7 | 1.3 | 3/3 | 11.7 | 0.9 | 3/3 | 11.9 | 0.2 | 3/3 |
| Eluate | | | | | | | | | |
| 3 μl | 12.1 | 0.2 | 3/3 | 13 | 1.2 | 3/3 | 12.5 | 1.3 | 3/3 |
| Eluate | | | | | | | | | |
| 0 μl | 12.3 | 0.3 | 3/3 | 12.3 | 0.3 | 3/3 | 12.3 | 0.3 | 3/3 |
| Eluate | | | | | | | | | |

TTA: time to amplification, SD: standard deviation.

### Example 4

This example illustrates the specific capture of an RNA template with Streptavidin PMP and shows the effect of using 2, 1 or 0 wash steps on the downstream RT-LAMP amplification reaction. Reducing or eliminating the wash steps is beneficial for the adaptation of the protocol to a simple cartridge format for home use. 36 simulated nasal matrix samples without any added template were processed as described in Example 1 up to the beginning of the wash steps. The Capture oligomers used are shown in Table 3. The samples were placed in the magnetic field. The first 12 samples were washed twice using 100 μl of each of the two wash buffers and the protocol described in Example 1. The next 12 samples were only washed once with 500 μl of a wash buffer comprising 10 mM Tris pH7.5, 150 mM sodium chloride, 1 mM EDTA and 0.01% Tween-20. The last 12 samples did not undergo a wash step. The samples were then eluted with 20 μl elution buffer as described in Example 1 and the 12 samples for each treatment group were pooled together. The pooled eluates were then tested for their inhibitory effect on a RT-LAMP reaction. For each treatment group, either 20 μl, 5 μl or 1 μl of the eluate pool was added to a 50 μl RT-LAMP reaction. The template was either 1000c, 100c or 50c of an in-vitro transcribed RNA corresponding to the N-gene of SARS-CoV-2. The RT-LAMP reactions contained 1× iB5 buffer (Optigene, UK), 3 mM magnesium sulfate, 1 μM Syto-9, 1 M Betaine, 8 U GspM3.0 (Optigene, UK), 0.5U AMV (Promega) and the RT-LAMP primers listed in Table 3. The reactions were incubated for 30 minutes at 65° C. in a CFX96 Touch Real-Time PCR Detection System thermocycler (Bio-Rad) with a read every 30 seconds.

TABLE 3
Capture oligomers and RT-LAMP primers used in Example 4

| Capture oligo | Sequence | Amount |
|---|---|---|
| capN_358R | Biotin-CAGCTTCTGGCCCAGTTCCTAGGTAGT | 8 pmoles |
| | (SEQ ID NO: 13) | |
| capN_886R | Biotin- | 8 pmoles |
| | AAAAATTTCCTTGTCTGATTAGTTCCTGGTCCC | |
| | (SEQ ID NO: 14) | |
| | | Amount |
| LAMP primers | Sequence | (μM) |
| Cv19-N2-F3 | ACACAAGCTTTCGGCAGAC (SEQ ID NO: 15) | 0.2 |
| Cv19-N2-B3 | CCAATTTGATGGCACCTGTG (SEQ ID NO: 16) | 0.2 |
| Cv19-N2-FIP1 | TGTGCAATTTGCGGCCAATGTTTGGTCCAGAACA | 1.4 |
| | AACCCAAG (SEQ ID NO: 17) | |
| Cv19-N2-BIP | CCAGCGCTTCAGCGTTCTTCGTAGGTCAACCACG | 1.4 |
| | TTCCCG (SEQ ID NO: 18) | |
| Cv19-N2-LF1 | CTGATTAGTTCCTGGTCCCCA (SEQ ID NO: 19) | 0.7 |
| Cv19-N2-LB1 | AATGTCGCGCATTGGCATGG (SEQ ID NO: 20) | 0.7 |

Table 4 shows the time to amplification for the LAMP reactions in the presence of the various samples. The samples with 2 wash steps or with 1 wash step caused no or very minimal inhibition of the RT-LAMP reactions. The samples without any wash step caused significant inhibition of the RT-LAMP reaction: the amplification was delayed in the presence of either 5 μl or 1 μl of the eluate in a 50 μl RT-LAMP reaction. We were however still able to successfully amplify 50c of RNA template in most of the samples, showing that the sensitivity was not greatly affected.

TABLE 4
Average time to amplification (AvgCq) of the RT-LAMP reactions
with 2, 1, or no washes in the presence of the various samples.

| | Eluent | | LAMP | AvgCq | | | #pos |
|---|---|---|---|---|---|---|---|
| | into | | RNA | (min- | | | by 15 |
| Condition | LAMP | | input | ute) | SD | #pos | min |
| 2 washes | 20 μl | (100%) | 1e3c | 6.6 | 0.7 | 2/2 | 2/2 |
| (100 μl wash | | | 100c | 7.9 | 0.1 | 2/2 | 2/2 |
| buffers S&T) | | | 50c | 10 | 1.7 | 4/4 | 4/4 |
| | 5 μl | (25%) | 1e3c | 6.1 | 0.4 | 2/2 | 2/2 |
| | | | 100c | 7.6 | 1.1 | 2/2 | 2/2 |
| | | | 50c | 9.1 | 1.7 | 8/8 | 8/8 |
| | 1 μl | (5%) | 1e3c | 6.1 | 0.4 | 2/2 | 2/2 |
| | | | 100c | 7.4 | 0.9 | 2/2 | 2/2 |
| | | | 50c | 7.7 | 0.7 | 4/4 | 4/4 |
| 1 wash | 20 μl | (100%) | 1e3c | 6.7 | 0.2 | 2/2 | 2/2 |
| (500 μl wash | | | 100c | 8 | 0.6 | 2/2 | 2/2 |
| buffer T+) | | | 50c | 9.7 | 1.7 | 4/4 | 4/4 |
| | 5 μl | (25%) | 1e3c | 6 | 0.1 | 2/2 | 2/2 |
| | | | 100c | 7.8 | 1 | 2/2 | 2/2 |
| | | | 50c | 8.2 | 1.2 | 8/8 | 8/8 |
| | 1 μl | (5%) | 1e3c | 6.7 | 0.5 | 2/2 | 2/2 |
| | | | 100c | 8.2 | 0 | 2/2 | 2/2 |
| | | | 50c | 12.6 | 7.6 | 4/4 | 3/4 |
| No washes | 20 μl | (100%) | 1e3c | | | 0/2 | 0/2 |
| | | | 100c | | | 0/2 | 0/2 |
| | | | 50c | | | 0/4 | 0/4 |
| | 5 μl | (25%) | 1e3c | 7.9 | 0.2 | 2/2 | 2/2 |
| | | | 100c | 19.1 | 2.8 | 2/2 | 0/2 |
| | | | 50c | 11.6 | 2.4 | 7/8 | 7/8 |
| | 1 μl | (5%) | 1e3c | 6.4 | 0.2 | 2/2 | 2/2 |
| | | | 100c | 7.6 | 0.2 | 2/2 | 2/2 |
| | | | 50c | 11 | 0.06 | 2/4 | 2/4 |
| LAMP | No eluent | | 1e3c | 6.1 | 0.4 | 2/2 | 2/2 |
| Standards and | | | 100c | 6.8 | 0.1 | 2/2 | 2/2 |
| Controls | | | 50c | 10.2 | 1.9 | 4/4 | 4/4 |
| | | | NTC | | | 0/2 | 0/2 |
| | 5 μl | (25%) | Neg | | | 0/2 | 0/2 |

### Example 5

This example shows that the inhibition of downstream RT-LAMP amplification by the samples can be minimized by altering the composition of the lysis/hybridization buffer, and in particular by lowering the detergent concentration to a concentration that is not inhibitory but is sufficient to successfully process a clinical sample. 30 simulated nasal matrix samples without any added template were processed with Streptavidin beads as described in Example 1 except that the composition of the lysis/hybridization was varied so that the final concentration of the detergent in the specific capture reactions was either 3% SDS (as in Example 1), 0.1% SDS, 1% Sarkosyl or 0.1% Sarkosyl. The sequences and amounts of the Capture oligomers are the same as in Table 3. The samples were either submitted to 2 washes with 100 μl of each of the 2 wash buffers described in Example 1 or were not submitted to any wash step. 6 replicates were prepared for each condition and the eluates for each condition were pooled. 20 μl, 10 μl, 5 μl, 2 μl or 1 μl of the eluate was used in 50 μl RT-LAMP reactions. Each reaction contained 1e3c SARS-COV-2 N-gene RNA. The RT-LAMP reactions were performed as described in Example 4. The results of the experiment are shown in Table 5. These results show that the samples submitted to 2 washes caused much less inhibition of RT-LAMP than the samples that weren't submitted to any wash step. The samples processed with lysis/hybridization buffers with either 0.1% SDS, 0.1% Sarkosyl or 1% Sarkosyl also caused less inhibition than the samples processed with the Lysis/Hybridization Buffer with 3% SDS. This indicates that SDS is strongly inhibitory to the RT-LAMP reaction and that reducing the SDS concentration of the lysis/hybridization buffer allows for a simplified wash protocol.

TABLE 5
Effect of the detergent composition of the Lysis/Hybridization Buffer and the number
of wash steps on the RT-LAMP amplification. AvgCq is the average time to amplification,
STDEV is the standard deviation and RFU % is a measure of the amplitude of the
fluorescent signal compared to that of the reaction without eluate.

| | | | RT-LAMP | | | | | | |
|---|---|---|---|---|---|---|---|---|---|
| | Specific capture | | RNA | Eluate | | Results | | | |
| | Detergent | Wash | input in | volume in | | AvgCq | | | |
| Condition | in Lys/Hyb | steps | RT-LAMP | LAMP | | (minutes) | STDEV | #positives | RFU % |
| 1 | 3% SDS | 2 wash | 1e3c | 20 | μl | 7.9 | 0.35 | 3/3 | 70% |
| | | steps | 1e3c | 10 | μl | 7.4 | 0.54 | 3/3 | 83% |
| | | | 1e3c | 5 | μl | 7 | 0.04 | 3/3 | 90% |
| | | | 1e3c | 2 | μl | 7.1 | 0.19 | 3/3 | 99% |
| | | | 1e3c | 1 | μl | 7.3 | 0.25 | 3/3 | 85% |
| 2 | 3% SDS | No | 1e3c | 20 | μl | N/A | | 0/3 | — |
| | | washes | 1e3c | 10 | μl | N/A | | 0/3 | — |
| | | | 1e3c | 5 | μl | N/A | | 0/3 | — |
| | | | 1e3c | 2 | μl | 13.4 | 0.73 | 3/3 | 23% |
| | | | 1e3c | 1 | μl | 12.1 | 2.09 | 3/3 | 36% |
| 3 | 0.1% SDS | No | 1e3c | 20 | μl | 17.1 | 0.54 | 2/3 | 13% |
| | | washes | 1e3c | 10 | μl | 13.3 | 1.76 | 3/3 | 51% |
| | | | 1e3c | 5 | μl | 8.5 | 0.4 | 3/3 | 86% |
| | | | 1e3c | 2 | μl | 8.6 | 0.42 | 3/3 | 96% |
| | | | 1e3c | 1 | μl | 7.8 | 0.35 | 3/3 | 94% |
| 4 | 1% | No | 1e3c | 20 | μl | N/A | | 0/3 | — |
| | Sarkosyl | washes | 1e3c | 10 | μl | 11.6 | 2.02 | 3/3 | 47% |
| | | | 1e3c | 5 | μl | 7.6 | 0.53 | 3/3 | 85% |
| | | | 1e3c | 2 | μl | 6.8 | 0.53 | 3/3 | 100% |
| | | | 1e3c | 1 | μl | 7.6 | 0.52 | 3/3 | 92% |
| 5 | 0.1% | No | 1e3c | 20 | μl | 15.6 | | 1/3 | 9% |
| | Sarkosyl | washes | 1e3c | 10 | μl | 11.7 | 0.93 | 3/3 | 68% |
| | | | 1e3c | 5 | μl | 8.5 | 0.55 | 3/3 | 98% |
| | | | 1e3c | 2 | μl | 7.6 | 0.2 | 3/3 | 100% |
| | | | 1e3c | 1 | μl | 7.6 | 0.14 | 3/3 | 96% |

| | | | | | | | | |
|---|---|---|---|---|---|---|---|---|
| Standards | None | 1e3c | 0 | μl | 6.9 | 0.25 | 3/3 | 102% |

| | | | | | | |
|---|---|---|---|---|---|---|
| 1e2c | 0 | μl | 7.6 | 0.51 | 3/3 | 103% |
| 5e1c | 0 | μl | 9.1 | 0.24 | 3/3 | 95% |
| 0 | 0 | μl | N/A | | 0/3 | — |

### Example 6

### Investigation of Alternative Detergents for the Lysis/Hybridization Buffer.

This example shows the ability of various detergents to clarify simulated nasal matrix (SNM) made of 5% type II mucin from porcine stomach, 1% blood, 15% glycerol, 137 mM NaCl, 10 mM Na₂HPO₄, 1.8 mM KH₂PO₄ and 2.7 mM KCl. 100 μl of SNM was mixed with 500 μl of various formulations of the Lysis/Hybridization buffer. The Lysis/Hybridization buffers tested all contained 25 mM Tris pH 8, 25 mM EDTA and 188 mM NaCl. In addition, they also contained either 1%, 0.5% or 0.1% of one of the following detergents: SDS, Sarkosyl, Triton X-100, Igepal CA-360, Tween-20 or CHAPS. The SNM and buffers were mixed together in a tube and the ability of the buffer to clarify the SNM was assessed visually. The samples were then placed at 95° C. for 10 minutes and inspected visually again. The results demonstrate that SDS at 1% and 0.5%, and Sarkosyl at either 1%, 0.5% or 0.1% in the Lysis/Hybridization buffer were able to partially clarify the SNM upon addition, whereas the samples with 0.1% SDS were clarified to a lesser extent but were clarified further after the heat step. Lysis/Hybridization buffers with 1% or 0.5% of either Triton X-100 or Igepal CA-630 hit cloud point whereas samples with 0.1% of these detergents were not clarified upon addition or after the heat step. Samples with 1%, 0.5% or 0.1% Tween-20 were not clarified upon contact but were clarified after the heat step. Samples with CHAPS were only very slightly clarified upon contact and were not clarified any further after the heat step.

### Example 7

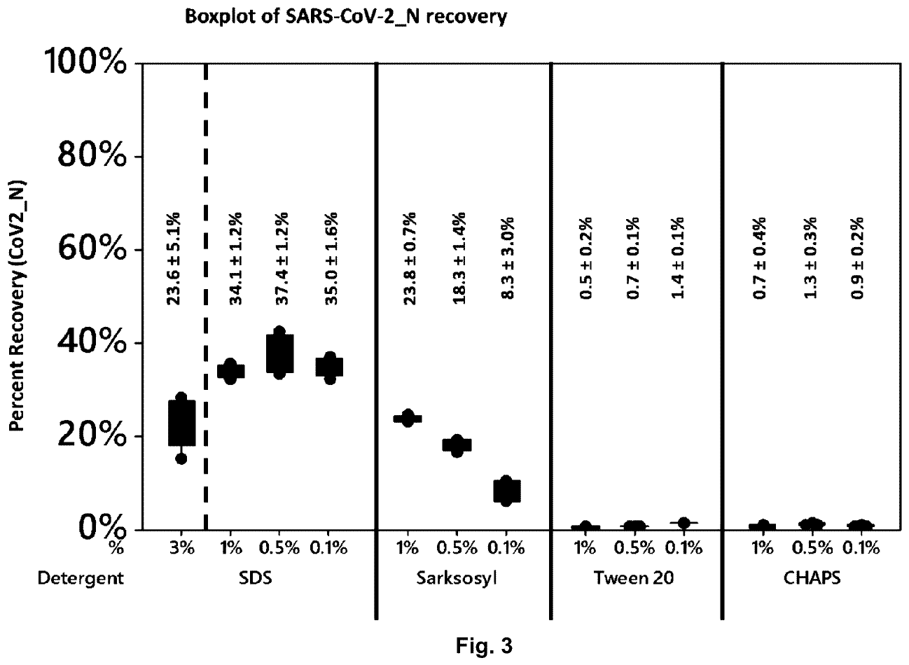

This example shows that Lysis/Hybridization buffers formulated with SDS and to a lesser extent with Sarkosyl perform much better in specific target capture than buffers formulated with either Tween-20 or CHAPS. Samples containing 100 μl simulated nasal matrix (formulated as in Example 6) and 1e6c or 0 c of template (SARS-COV-2 N-gene in-vitro-transcribed RNA) were used in specific target capture as described in Example 1, except that different formulations of the lysis/hybridization buffer containing various detergents were used. The Lysis/Hybridization solutions all contained 25 mM Tris pH 8, 25 mM EDTA and 188 mM NaCl. In addition, they contained either 3% SDS, 1% SDS, 0.5% SDS, 0.1% SDS, 1% Sarkosyl, 0.5% Sarkosyl, 0.1% Sarkosyl, 1% Tween-20, 0.5% Tween-20, 0.1% Tween-20, 1% CHAPS, 0.5% CHAPS, or 0.1% CHAPS. The sequences of the capture oligomers are listed in Table 3. The amount of RNA recovered in the eluates after the specific target capture was quantified by qRT-PCR. The qPCR reactions contained 10 μl 2× qScript XLT 1-Step RT-qPCR ToughMix (QuantaBio), 1 μM SYBR Green (ThermoFisher), 200 nM of each primer N_653F: CTCTTGCTTTGCTGCTG (SEQ ID NO:21) and N_852R: TCCTTGGGTTTGTTCTGG (SEQ ID NO:22), and 5 μl eluate sample for a total volume of 20 μl. The reactions were incubated for 5 minutes at 55° C., 5 minutes at 95° C., followed by 40 cycles of 10 seconds at 95° C., 10 seconds at 55° C. and 20 seconds at 72° C., with a fluorescent read at the end of each cycle. The % of RNA that was recovered after the specific capture was quantified with a standard curve. The results show that SDS performed better than any of the other detergents tested, and that concentrations of SDS of 1%, 0.5% or 0.1% performed better than 3% SDS. Sarkosyl performed less well than SDS. Lysis/Hybridization buffer formulations with either Tween-20 or CHAPS were ineffective at capturing the target RNA. The results are shown in FIG. 3.

### Example 8

### Effect of Fragmentation of the Genomic DNA on Specific Capture.

This example demonstrates that the specific capture of genomic DNA with Streptavidin beads can be improved by fragmenting the genomic DNA template either by sonication or by digestion with a restriction endonuclease (RE). Genomic DNA from Neisseria gonorrhoeae (strain FA1090, Genbank NC_002946, ATCC 700825D-5) was either used intact, sonicated for 5s at power 5/10 using Misonix Sonicator 3000® or digested with restriction endonucleases as follows: 116 ng genomic DNA was digested in a 50 μl reaction for 1 hour at 37° C. with 10 units of HindIII (NEB) and 10 units of ScrFI (NEB) in 1× buffer NEB2.1 (NEB, USA), followed by heat-inactivation at 80° C. for 30 minutes. This reaction was diluted and used in the specific capture reaction without further purification. Capture of 1e5c of the genomic DNA (intact, sonicated or digested) was compared to 1e5c of a synthetic double-stranded DNA template (963 bp in length, encoding the ISNgo2 gene) and each condition was run in triplicate. The standard capture experiments were performed as described in Example 1, using the capture oligomer mixture listed in Table 1. The amount of DNA recovered after the specific target capture was quantified by qPCR. Each qPCR reaction contained 1× PerfeCTa qPCR ToughMix (QuantaBio), 500 nM primer F370: ACGAGCAATACAGGCTTTCA (SEQ ID NO:23), 500 nM primer R479: TTGGCCGCTTCTTCATCTT (SEQ ID NO:24) and 1 μM SYBR Green. The reactions were incubated at 95° C. for 10 minutes, followed by 40 cycles of 95° C. for 10 seconds, 60° C. for 10 seconds and 72° C. for 20 seconds with a fluorescence read at the end of each cycle. Each sample was quantified using a standard curve made from the same DNA template.

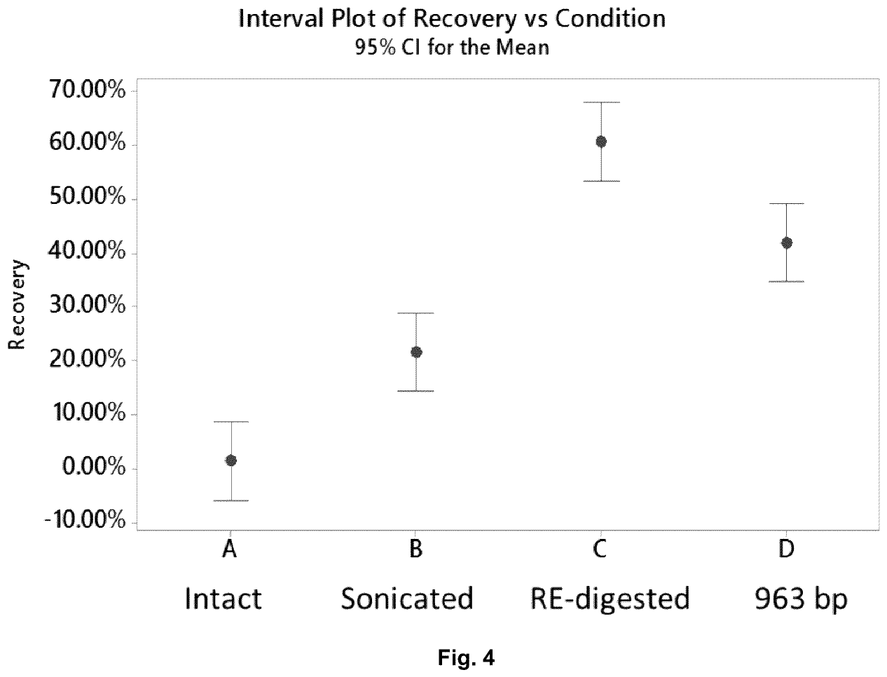

The results are shown in FIG. 4 and Table 6. Intact genomic DNA was not captured effectively with only 1.4% DNA recovered. Sonicating the genomic DNA improved the capture efficiency to 21.6% whereas digesting the genomic DNA with the restriction endonucleases increased the recovery rate to 41.9%. The short synthetic DNA fragment was captured with a recovery rate of 60.6%.

TABLE 6
Percent recovery of intact DNA, sonicated DNA, a 963-bp
synthetic DNA fragment, and RE-digested DNA.

| | % DNA |
|---|---|
| Template | recovery |
| 1e5c intact genomic DNA | 1.36% |
| 1e5c sonicated genomic DNA | 21.56% |
| 1e5c DNA fragment (963 bp) | 60.63% |
| 1e5c RE-digested genomic DNA | 41.86% |

### Example 9

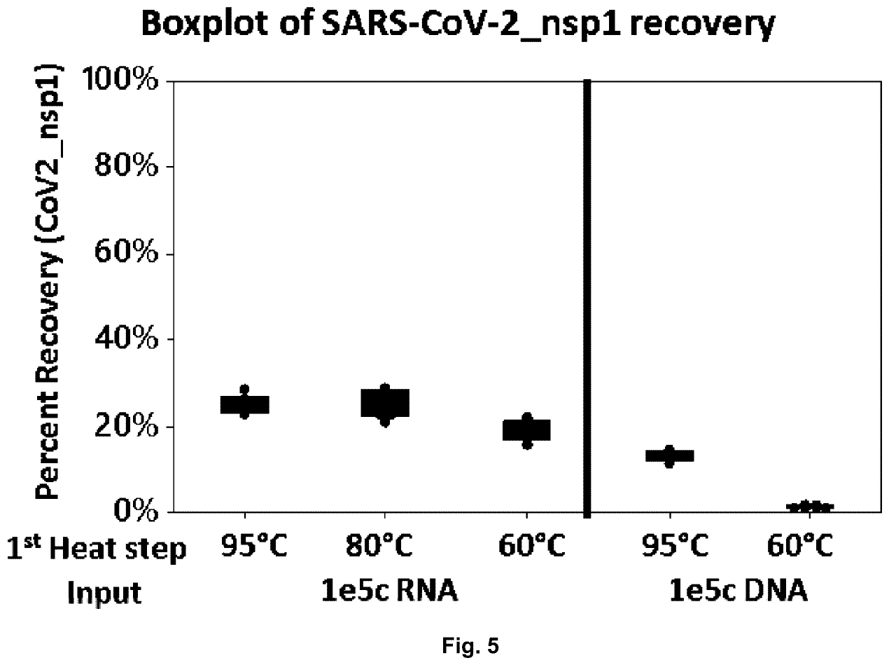

This example demonstrates that the initial denaturation step can be performed at a lower temperature with minimal effect on RNA template recovery, but that specific capture of DNA involves a denaturation step at 95° C. The template used was 1e5c of an in-vitro transcribed RNA corresponding to the nsp1 gene of SARS-COV-2 (540 nucleotides) or 1e5c of a double-stranded synthetic DNA fragment of 627 bp containing the nsp1 gene. The samples comprised the template RNA or DNA and 100 μl simulated nasal matrix (described in Example 6). The specific capture was performed as described in Example 1 except that the first heating step was either 10 minutes at 95° C., 10 minutes at 80° C. or 10 minutes at 60° C. The capture oligomers used in this experiment are listed in Table 7. Each condition was run in triplicate. The captured RNA was quantified by RT-qPCR using the method described in Example 7, with the primers listed in Table 7. The captured DNA was quantified by qPCR using the method described in Example 8, with the primers listed in Table 7. The results show that lowering the denaturation to 80° C. has no effect on RNA capture efficiency, whereas lowering the denaturation temperature to 60° C. has only a small effect. Lowering the denaturation temperature to 60° C. on the other hand greatly reduces the capture efficiency for DNA templates (Table 8 and FIG. 5).

TABLE 7
Capture oligomers and qPCR primers used in Example 9.

| Capture | | |
|---|---|---|
| Oligos | Sequence | Amount |
| nsp1_88R | Biotin- | 8 pmoles |
| | AAAAATCACGTACGAGCACGTCGCGAACCTGTA | |
| | (SEQ ID NO: 25) | |
| nsp1_350R | Biotin- | 8 pmoles |
| | AAAAATGCCACTGGTATTTCGCCCACATGAGG | |
| | (SEQ ID NO: 26) | |
| qPCR | | |
| primers | Sequence | Concentration |
| 145F | GGCACTTGTGGCTTAGTAGAA (SEQ ID NO: 27) | 500 nM |
| 270R | TGCTACCAGCTCAACCATAAC (SEQ ID NO: 28) | 500 nM |

TABLE 8
Percent recovery of the nsp1 gene of SARS-CoV-2 RNA
or DNA with various denaturation temperatures.

| Initial denaturation step | Template | % Recovery | STDEV |
|---|---|---|---|
| 10 minutes at 95° C. | 1e5c RNA | 25.00% | 1.80% |
| 10 minutes at 80° C. | 1e5c RNA | 25.80% | 3.70% |
| 10 minutes at 60° C. | 1e5c RNA | 18.70% | 2.60% |
| 10 minutes at 95° C. | 1e5c DNA | 13.10% | 1.20% |
| 10 minutes at 60° C. | 1e5c DNA | 1.50% | 0.40% |

### Example 10 (Prophetic)

This example demonstrates the isolation of SARS-COV-2 RNA with rapid specific target amplification protocol using a magnetic field followed by RT-LAMP amplification directly from the magnetic beads. In this example, RNA amplification is further improved by modifying the Oligo-dT beads to have one or more inverted dT nucleotides on the 3′ end or an L-DNA analog on the 3′ end. For example, 5′-TTTTTTTTTTTTTT/3InvdT/-3′ (SEQ ID NO:97), 5′-TTTTTTTTTTTTTT/L-DNA dT/-3′ (SEQ ID NO:98). This leads to improved amplification by preventing a polymerase enzyme from engaging with the 3′ end of the Oligo-dT sequence on the bead and thereby freeing it up to engage and amplify the target of interest. RNA is captured and amplified in the same manner as described in Example 19, except that off-bead amplification is improved with the modification of the Oligo-dT sequence.

### Example 11

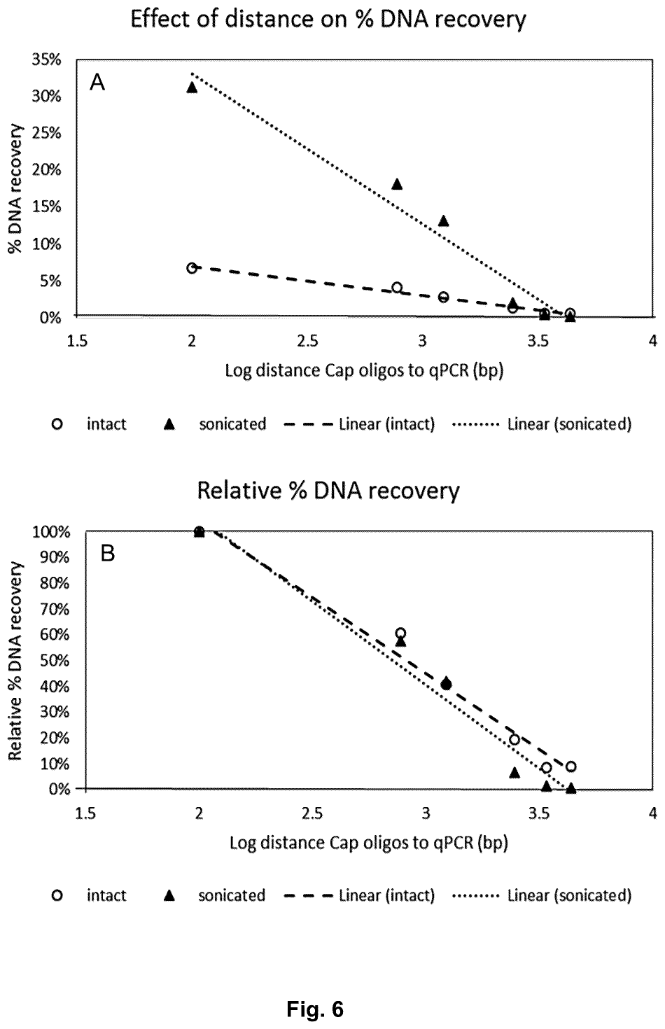

Improved amplification of a target sequence can be achieved by designing capture oligomers to bind to the template relatively close to the amplification target sequence. This example investigates how close the amplification target sequence has to be relative to the capture oligomers to effectively detect the captured DNA. The template used was genomic DNA from Mycobacterium tuberculosis strain H37Rv (ATCC #25618), either intact or sonicated for 3 times 5 seconds at power 5/10 using Misonix Sonicator 3000®. Each sample comprised 1e5c DNA in 600 μl of NaCl 300 mM. The sample was mixed with 200 μl of 4× lysis/hybridization buffer (100 mM Tris pH8, 12% SDS and 100 mM EDTA) and 2.5 pmoles of each of the 2 capture oligomers listed in Table 10. The samples were incubated for 10 minutes at 95° C. and 30 minutes at 60° C., then 200 μg of washed Streptavidin beads were added and the samples were incubated for 30 minutes at 45° C. with constant agitation. The samples were washed with 1 ml wash buffer (10 mM Tris pH 8.0 and 0.01% Tween-20), re-suspended in 50 μl Tris pH 7.5 and incubated for 3 minutes at 75° C. The samples were then placed in a magnetic field and the eluates were collected for qPCR amplification. A series of qPCR primer pairs were designed at various distances from the binding sites for the capture oligomers on the DNA template. The qPCR amplifications were as described in Example 8, using the primers shown in Table 10. A separate standard curve was made for each primer pair. The results are shown in Table 11 and FIG. 6. The results demonstrate that the % DNA recovery decreases with the distance between the annealing sites for the capture oligomers and the qPCR primers, suggesting that the DNA that is captured onto the PMP is made of relatively short fragments. The sonicated DNA is captured more effectively than the intact genomic DNA when the qPCR primer and capture oligomer binding sites are located in close proximity, but the % recovery drops below that of intact DNA when the qPCR primers are located more than 2000 bp away from the capture oligomers, suggesting that most DNA fragments after sonication are shorter than 2000 bp.

TABLE 10
Capture oligomers and qPCR primers used in Example 11.

| Capture oligo | Sequence | Amount |
|---|---|---|
| TBkatG_capF3 | Biotin- | 2.5 |
| | AAAAAATGGGTCTTACCGAAAGTGTGACCGCC (SEQ | pmoles |
| | ID NO: 29) | |
| TBkatG_capR2 | Biotin- | 2.5 |
| | AAAAAGCATCGAGGTCGTATGGACGAACACCC | pmoles |
| | (SEQ ID NO: 30) | |
| | | Distance |
| | | between Cap |
| | | oligos and |
| qPCR primers | Sequence | qPCR |
| TB-katG-F2 | CGTCCTTGGCGGTGTATT (SEQ ID NO: 31) | 99 |
| TB-KatG-R2 | GGACAACAGTTTCCTCGAGAT (SEQ ID NO: 32) | |
| TB-katG-1645F | TGTGCCGCCATAGAGAAAG (SEQ ID NO: 33) | 777 |
| TB-katG-1750F | AGGATTCCACGTCGGTTTG (SEQ ID NO: 34) | |
| TB-katG-2116F | GCGCTTGTCGAGGTCTATG (SEQ ID NO: 35) | 1237 |
| TB-katG-2210F | AACCTGTCGAGGTTCATCAC (SEQ ID NO: 36) | |
| TB-katG-3333F | ACCGAAGACGGCAATTGAT (SEQ ID NO: 37) | 2480 |
| TB-katG-3453R | GTTACCCATCCGCTTCCAATA (SEQ ID NO: 38) | |
| TB-katG-4231F | TGATCAACTGTGCTGGTCTG (SEQ ID NO: 39) | 3364 |
| TB-katG-4337R | CAGTTGCTCTAGACCTGGATTG (SEQ ID NO: 40) | |
| TB-katG-5268F | TGGCAAGCAGTGTGAGTAAG (SEQ ID NO: 41) | 4380 |
| TB-katG-5353R | CGCACACTAGAGCCACAAA (SEQ ID NO: 42) | |

TABLE 11
Percent recovery of intact and sonicated genomic
DNA with various distances between the capture
oligomer and the qPCR primer binding sites.

| | Intact | Sonicated |
|---|---|---|
| Distance between | genomic DNA | genomic DNA |
| Cap oligos and qPCR | % DNA recovery | % DNA recovery |
| 99 | 6.70% | 31.30% |
| 777 | 4.07% | 18.10% |
| 1237 | 2.73% | 13.10% |
| 2480 | 1.31% | 2.04% |
| 3364 | 0.56% | 0.47% |
| 4380 | 0.60% | 0.21% |

### Example 12

### Effect of the Length of the DNA Template on the Efficiency of DNA Capture.

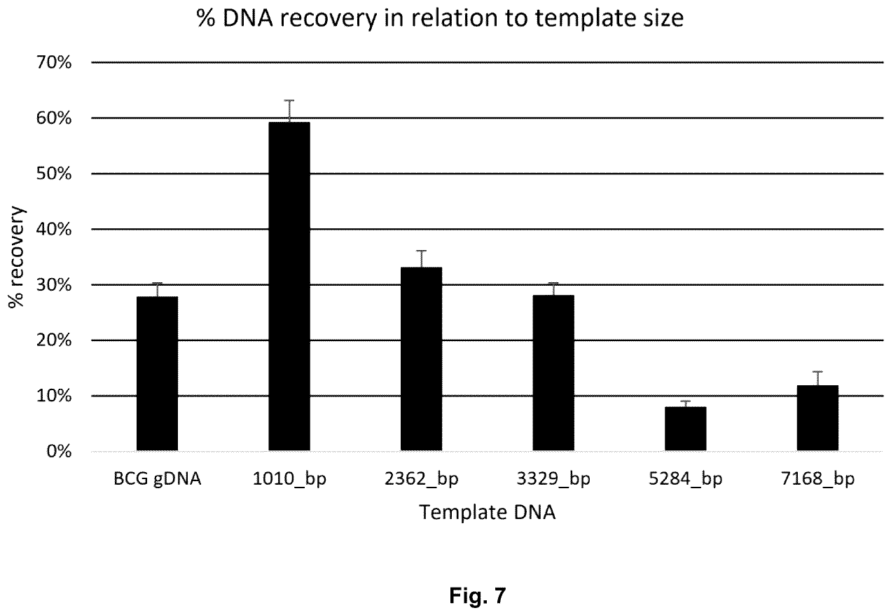

This example shows that short DNA fragments are captured more effectively than longer ones. DNA fragments of various pre-determined sizes were generated by PCR, quantified and used as templates in a specific target capture experiment. The % DNA captured was determined by qPCR. All the templates were captured using the same mixture of capture oligomers and quantified using the same qPCR primers. The PCR reactions to generate the DNA templates contained 1× Q5 Buffer, 1× High GC Enhancer, 0.2 mM dNTP, 500 nM forward primer, 500 nM Reverse primer and 2 units Q5 DNA polymerase (NEB, USA) in a 50 μl reaction. The reactions were run for 3 minutes at 95° C., followed by 35 cycles of 10 seconds at 95° C., 30 seconds at 65° C., and 2 minutes (fragments <3000 bp) or 6 minutes (fragments >3000 bp) at 72° C., followed by a final extension step of 10 minutes at 72° C. The sequence of the PCR primers is shown in Table 12 and the length of the fragments they generate in Table 13. The fragments were visualized on a 0.8% agarose gel and quantified using a Quant-iT PicoGreen dsDNA Assay kit (ThermoFisher) according to the manufacturer's directions. 1e6c of each PCR fragment or 1e6c of Mycobacterium bovis BCG genomic DNA (Strain TMC1011, ATCC #35734) were used as templates in the specific target capture reactions, which were performed as described in Example 11 with the capture oligomers mixture shown in Table 12 (1.4 pmoles of each capture oligomer for a total of 20 pmoles). Each capture reaction was done in triplicate. The % DNA recovery was determined by qPCR using 500 nM each of primers TB-rpoB-F3 and TB-rpoB-R3: the reaction conditions were as described in Example 8 and the primer sequences are shown in Table 12. The results are shown in Table 13 and in FIG. 7. The results show that longer fragments of DNA are captured less effectively than shorter fragments. Genomic DNA is captured about as effectively as fragments that are 2000 to 3000 bp long.

TABLE 12
Capture oligomers, PCR primers and qPCR primers used in Example 12.

| Capture oligo | Sequence |
|---|---|
| TBahpC_capR | Biotin-AAAAAGCGAGCGGTGGTCGGAAGCCGGTCTCG (SEQ ID NO: 43) |
| TBahpC_capF | Biotin-AAAAAGTGGCGGGTGGTGTTCTTTTGGCCGAA (SEQ ID NO: 44) |
| TBembB_capR | Biotin-AAAAAGCCCGACGGGTCCTTGAGCCCGACGAAG (SEQ ID NO: 45) |
| TBembB_capF | Biotin-AAAAACTCACACCGGCGGCGCTGGCCGTCGTT (SEQ ID NO: 46) |
| TBinhA_capR | Biotin-AAAAAGCGGGTGTATTGACGCCCTACGAATTGC (SEQ ID NO: 47) |
| TBinhA_capF | Biotin-AAAAAGCACAGCGGCTGGCTGCCGACGGCCAC (SEQ ID NO: 48) |
| TBkatG_capF3 | Biotin-AAAAAATGGGTCTTACCGAAAGTGTGACCGCC (SEQ ID NO: 49) |
| TBkatG_capR2 | Biotin-AAAAAGCATCGAGGTCGTATGGACGAACACCC (SEQ ID NO: 50) |
| TBpncA_CapR1 | Biotin-AAAAATCCCAGTCTGGACACGTCGGCAATCG (SEQ ID NO: 51) |
| TBpncA_CapF1 | Biotin-AAAAACCTTGGTTGCCACGACGTGATGGTAGT (SEQ ID NO: 52) |
| TBrpoB_capF4 | Biotin-AAAAAGCGATCACACCGCAGACGTTGATCAAC (SEQ ID NO: 53) |
| TBrpoB_capR3 | Biotin-AAAAAAAGTGGTCGATGTCGTCGGTTTCCACC (SEQ ID NO: 54) |
| TB-embBpr- | Biotin-AAAAACGGTTAAGGGCGGTGAGGATGCG (SEQ ID NO: 55) |
| CapR6 | |
| TB-embBpr- | Biotin-AAAAAATCGCACGCCTAGCAGCCGTCGT (SEQ ID NO: 56) |
| CapF6 | |
| PCR primers | Sequence |
| 19-111_TB-rpoB-dis2_F | GACGAGTGCAAAGACAAGGA (SEQ ID NO: 57) |
| 19-113_TB-rpoB-dis3_F | TCGTCTGAGGTGGACTACAT (SEQ ID NO: 58) |
| 19-114_TB-rpoB-dis3_R | GCTCCAGGAAGGGAATCATC (SEQ ID NO: 59) |
| 19-115_TB-rpoB-dis4 F | CGTGTGTATGTGGCTCAGAA (SEQ ID NO: 60) |
| 19-116_TB-rpoB-dis4_R | GGAACGGCATGTCCTCAA (SEQ ID NO: 61) |
| 19-117_TB-rpoB-dis5_F | TGCTCGACGTCAACTTCTTC (SEQ ID NO: 62) |
| 19-118_TB-rpoB-dis5_R | GTTGATCGTCTCCGGCTTT (SEQ ID NO: 63) |
| 19-119_TB-rpoB_dis6_F | AGATCGAGGCCGAGCTATT (SEQ ID NO: 64) |
| 19-120_TB-rpoB-dis6_R | TGCTTGTTGACGAACGGATAA (SEQ ID NO: 65) |
| 19-121_TB-rpoB-dis7_F | ATCCCGTCGTATGAGGATCA (SEQ ID NO: 66) |
| 19-122_TB-rpoB-dis7_R | TAGTCGCTGTAGCCGTAGT (SEQ ID NO: 67) |
| qPCR primers | Sequence |
| 19-075_TB-rpoB-F3 | CACACCGCAGACGTTGAT (SEQ ID NO: 68) |
| 19-076_TB-rpoB-R3 | GGTTGTTCTGGTCCATGAATTG (SEQ ID NO: 69) |

TABLE 13
Percent recovery of DNA with various fragment sizes.

| | DNA input | | | STC |
|---|---|---|---|---|
| | Fw | Rv | Fragment | % DNA |
| | primer | primer | size | recovery |
| 1e6c PCR | TB-rpoB-dis2F | TB-rpoB-R4 | 1010 bp | 59.33% |
| fragment | | | | |
| 1e6c PCR | TB-rpoB-dis2F | T-rpoB-dis4R | 2362 bp | 33.20% |
| fragment | | | | |
| 1e6c PCR | TB-rpoB-dis2F | T-rpoB-dis5R | 3329 bp | 28.13% |
| fragment | | | | |
| 1e6c PCR | TB-rpoB-dis2F | T-rpoB-dis6R | 5284 bp | 8.07% |
| fragment | | | | |
| 1e6c PCR | TB-rpoB-dis2F | T-rpoB-dis7R | 7168 bp | 11.91% |
| fragment | | | | |
| 1e6c BCG | | | | 27.87% |
| genomic DNA | | | | |

### Example 13

This example demonstrates that the specific target capture protocol can be shortened to a total time of 20 minutes and still extract a useful amount of DNA from a blood sample. The samples were assembled in 200 μl PCR tubes and the heat incubations were done in a thermocycler to speed up the process. Each sample was done in triplicate. The samples were 1e5c of an artificial double-stranded DNA fragment comprising the uidA gene of E. coli in either 100 μl NaCl 300 mM or 100 μl blood. The samples were mixed with 33.3 μl of a 4× hybridization buffer (100 mM Tris pH 7.5, 100 mM EDTA, 12% SDS), 2 pmoles each of the capture oligomers EC-uidA-754R: Biotin-GTTCATAGAGATAACCTTCACCCGGTTGCC (SEQ ID NO:70) and EC-uidA-887F: Biotin-TTGGTCGTCATGAAGATGCGGATTTGCG (SEQ ID NO:71) and incubated for 3 minutes at 95° C. and 2 minutes at 60° C. 200 μg of streptavidin beads were added, the samples were mixed and incubated for 2 minutes at 45° C. The samples were then processed as described in Example 1. The eluted DNA was quantified by qPCR as described in Example 8, but using the primers EC_uidAF: CCAAAAGCCAGACAGAGTGTGAT (SEQ ID NO:72) and EC uidAR: AGCCAGTAAAGTAGAACGGTTTGT (SEQ ID NO:73). The results show that the shortened protocol still extracts an amount of DNA that would be useful for a point-of-care diagnostic device, and that the DNA recovery from the blood samples was more than half of that from the NaCl samples.

TABLE 14
Percent recovery of DNA with shortened
specific target capture protocol.

| | % DNA | |
|---|---|---|
| Sample | recovery | STDEV |
| 1e5c EC_uidA dsDNA in | 29.40% | 6.20% |
| 300 mM NaCl | | |
| 1e5c EC_uidA dsDNA in | 16.80% | 3.60% |
| blood | | |

### Example 14

Simple Devices to Remove the Lys/Hyb Buffer from the PMP by Filtration.

One possible iteration of a diagnostic device using specific target capture would have the lysis/hybridization solution removed from the magnetic particles by filtration on a membrane instead of using a magnetic field, and the captured template would be amplified directly on the PMP retained on the membrane without an elution step. Filtration membranes of several different types and with different pore sizes were tested for their ability to rapidly filter viscous mucin-containing samples, to retain the beads on their surface, and their compatibility with LAMP amplification reactions. In this example, simple size exclusion devices were made to assess the flow rate of viscous mucin-containing samples through different membrane types using only gravity and capillary action (FIG. 8A). The devices comprised a disk of the membrane of interest of a diameter of 11 mm placed on top of 4 rectangles (3.5 cm×5 cm) of GB003 filter paper. A cut 1 ml blue pipette tip was placed on the membrane with the cut end facing up and firm pressure was applied. The sample was placed in the blue tip reservoir. All the membranes were purchased from Sterlitech. The types of membrane tested were 1) cellulose acetate (CA) membranes with pore sizes of 0.2 μm, 0.45 μm, 0.65 μm, 0.8 μm or 1.2 μm, 2) mixed cellulose esters membranes (MCE) with pore sizes of 0.2 μm, 0.45 μm, 0.65 μm, 0.8 μm or 1.0 μm, 3) a polyacrylonitrile membrane (PAN) with a pore size of 0.2 μm, 4) polycarbonate track etched (PCTE) membranes with pore sizes of 0.1 μm, 0.2 μm, 0.4 μm, 0.6 μm or 0.8 μm, 5) polyethersulfone membranes (PES) with pore sizes of 0.1 μm, 0.2 μm, 0.45 μm, 0.65 μm or 0.8 μm, 6) polyester track etched membranes (PETE) with pore sizes of 0.1 μm, 0.2 μm, 0.4 μm, 0.8 μm or 1.0 μm, and 7) polyvinylidene membranes (PVDF) with pore sizes of 0.2 μm or 0.45 μm. In an initial experiment, 50 μg Sera-Mag Oligo (dT)₁₄ PMP from Cytiva were mixed with 200 μl wash buffer and filtered through the devices. The membranes and the filter paper were inspected for the ability of the membranes to retain the beads on their surface. The results are shown in FIG. 8B and show that all the membranes effectively retained the beads, with no visible beads on the underlying filter paper. In a second experiment, mock vaginal fluid samples were passed through the device to assess the flow rate of the different membranes. The samples comprised 500 μl D1.2× buffer and 100 μl Simulated Vaginal Fluid (SVF) as described in Example 3. They were heated for 10 minutes at 95° C., then 10 minutes at 60° C. then left to cool to room temperature and placed in the device. The time it took for the samples to filter through by gravity and capillary action was measured. Once all the liquid had gone through, the membrane disk was carefully lifted with tweezers and placed on a fresh stack of four filter papers and 600 μl wash buffer (10 mM Tris pH7.5, 150 mM sodium chloride, 1 mM EDTA and 0.01% Tween-20) was added to the blue tip sample reservoir and allowed to flow through. The time it took for the wash buffer to filter through was also measured. Table 15 shows the flow rate of the samples through the different membranes and FIGS. 8A and 8B show the filtration devices and the membranes after filtration of 5 μg beads. The results show that most membranes retained the beads effectively: only the PAN membrane had some leakage around the edges. The PES, MCE, PETE and CA membranes had acceptable flow rates.

TABLE 15
Flow rate of the samples through the different membranes.

| | | | | Filtration | | |
|---|---|---|---|---|---|---|
| | Pore | Filtration | | time wash | | Bead |
| Membrane | size (μM) | time sample | | buffer | | retention |
| CA | 0.2 | Approx. 3 min | | 2 min 18 sec | | Not done |
| | 0.45 | 1 min 30 sec | | 44 | sec | Not done |
| | 0.65 | 1 min 30 sec | | 52 | sec | Not done |
| | 0.8 | 1 min 22 sec | | 40 | sec | Remain on |
| | | | | | | membrane |
| | 1.2 | 1 min 12 sec | | 38 | sec | Remain on |
| | | | | | | membrane |
| MCE | 0.2 | 2 min 35 sec | | 2 min 54 sec | | Not done |
| | 0.45 | 2 min 56 sec | | 2 min 45 sec | | Not done |
| | 0.65 | 4 min 15 sec | | 1 min 30 sec | | Not done |
| | 0.8 | 2 min 26 sec | | 1 min 13 sec | | Not done |
| | 1 | 49 | sec | 31 | sec | Remain on |
| | | | | | | membrane |
| PAN | 0.2 | 45 | sec | 38 | sec | leak around |
| | | | | | | edges |
| PCTE | 0.1 | Didn't go | | Not done | | Not done |
| | | through | | | | |
| | 0.2 | >9 | min | Not done | | Not done |
| | 0.4 | 7 min 35 sec | | 4 min 40 sec | | Not done |
| | 0.6 | 6 min 15 sec | | 1 min 10 sec | | Remain on |
| | | | | | | membrane |
| | 0.8 | 2 | min | 36 | sec | Remain on |
| | | | | | | membrane |
| PES | 0.1 | Approx. 9 min | | 6 min 50 sec | | Not done |
| | 0.2 | Approx. 4 min | | 1 min 55 sec | | Not done |
| | 0.45 | Approx. 3 min | | 1 | min | Not done |
| | 0.65 | Approx. 1 min | | 33 | sec | Not done |
| | 0.8 | 35 | sec | 20 | sec | Remain on |
| | | | | | | membrane |

### Example 15

Compatibility of the Filtration Membranes with Real-Time LAMP or RT-LAMP Amplification.

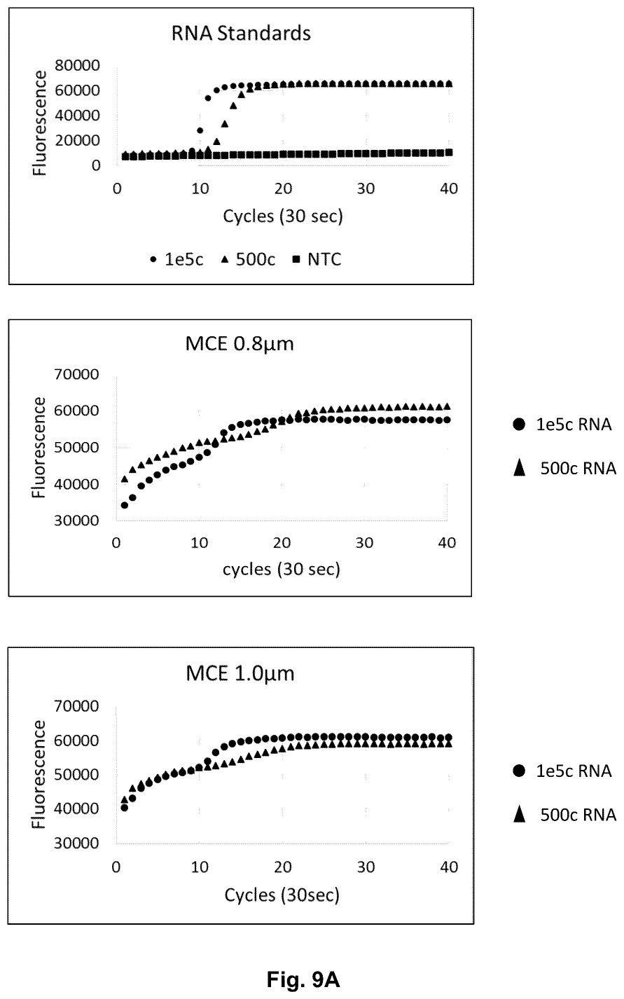

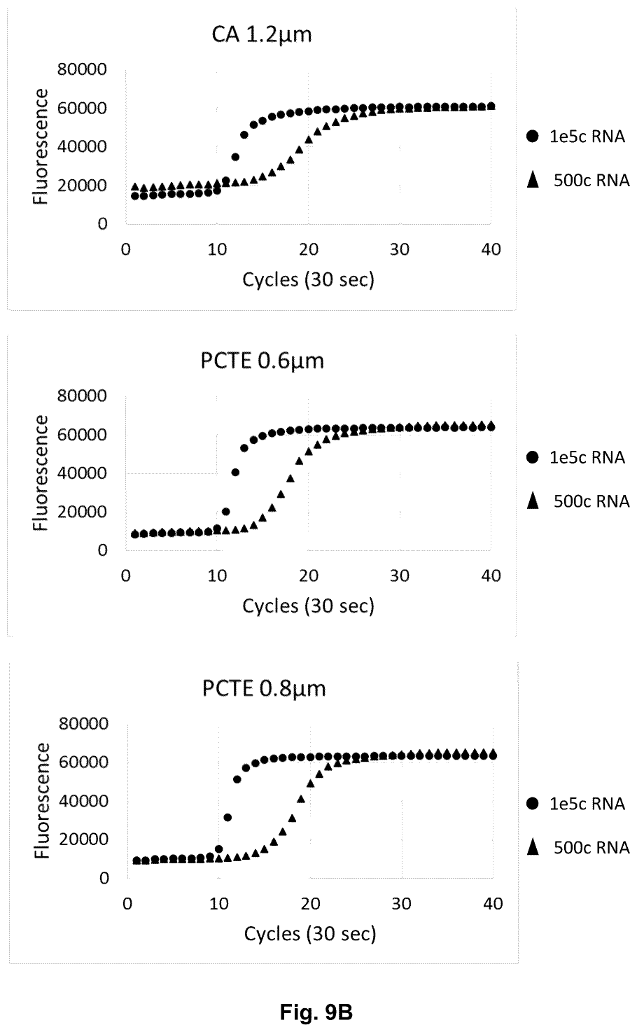

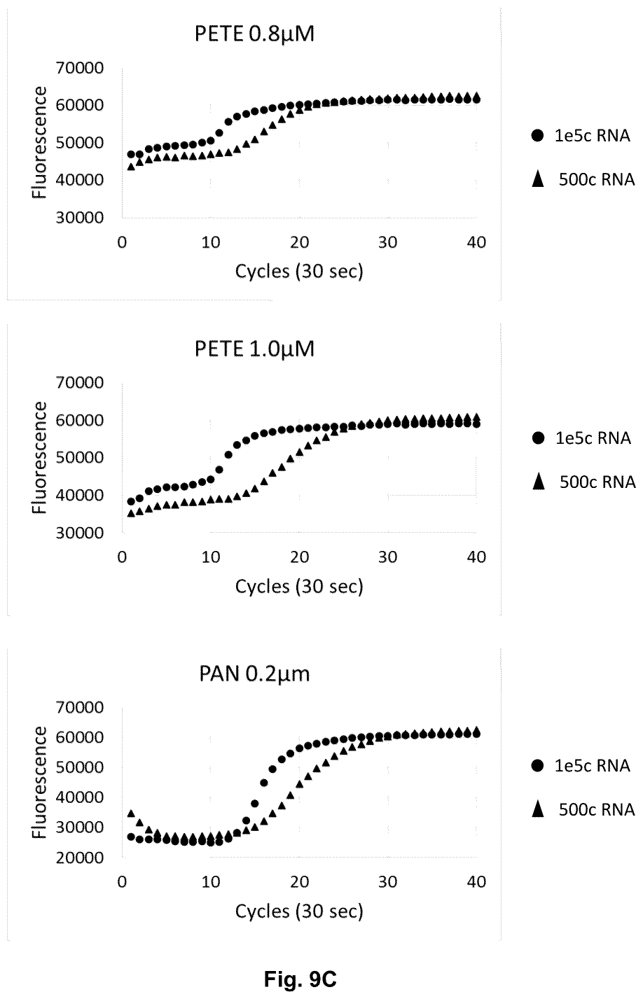

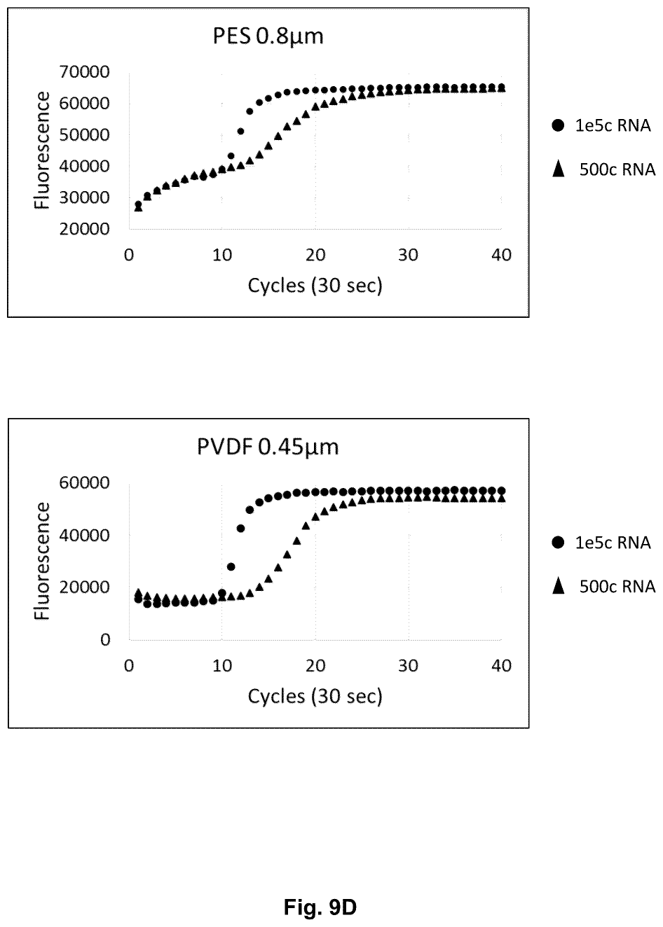

One possible iteration of a diagnostic device using specific target capture (STC) would have the lysis/hybridization solution removed from the magnetic particles by filtration instead of using a magnetic field, and the captured template amplified directly on the PMP retained on the membrane without an elution step. This example investigates the effect of the membranes on a RT-LAMP reaction. 7 mm membrane disks were cut from each membrane and placed in PCR tubes containing 50 μl of a RT-LAMP reaction. The RT-LAMP reactions contained either 1e5c or 500c of an in-vitro transcribed RNA of the N-gene of SARS-COV-2, 1× iB5 buffer (Optigene, UK), 3 mM magnesium sulfate, 1 μM Syto-9, 1 M Betaine, 8 U GspM3.0 (Optigene, UK), 0.5 U AMV (Promega) and the RT-LAMP primers listed in Table 16. The reactions were incubated for 30 minutes at 65° C. in a MxPro3005P thermocycler (Stratagene) with a read every 30 seconds. Each condition was performed in duplicate. Table 17 shows the membrane used, the time to amplification (Cq) of the RT-LAMP reactions and the increase in relative fluorescence for each sample type. FIGS. 9A-9D show the data for the amplification curves for each membrane type. The results show that the fluorescent properties of the membranes are very different: some, like the PCTE membranes, have a very low background fluorescence and so are very compatible with real-time monitoring: they make good baselines with a big increase in fluorescence during amplification. Their flow rate however is too low to accommodate viscous samples (Table 15), making them poor candidates for a quick diagnostic device. Other membranes, such as the MCE membranes, have a very high and increasing initial fluorescence which makes them incompatible with real-time monitoring. The CA membranes have a favourable amplification profile and a good flow rate.

TABLE 16
RT-LAMP primers used in Example 15.

| | | concentration |
|---|---|---|
| LAMP primers | Sequence | (μM) |
| Cv19N-F3_501F | GCCAAAAGGCTTCTACGCA (SEQ ID NO: 74) | 0.2 |
| Cv19N-B3_740R | GTGACAGTTTGGCCTTGTTG (SEQ ID | 0.2 |
| | NO: 75) | |
| Cv19N-FIP_539F | TCCCCTACTGCTGCCTGGAGGTCAAGCCTC | 1.4 |
| | TTCTCGTTCC (SEQ ID NO: 76) | |
| Cv19N-BIP_720R | CAATGGCGGTGATGCTGCTCTTTGTTGGCC | 1.4 |
| | TTTACCAGACA (SEQ ID NO: 77) | |
| Cv19N-LF_579R | ACTGTTGCGACTACGTGATG (SEQ ID | 0.7 |
| | NO: 78) | |
| Cv19N-LB_677F | GATTGAACCAGCTTGAGAGC (SEQ ID | 0.7 |
| | NO: 79) | |

TABLE 17
Membrane used, time to amplification (Cq) of the
RT-LAMP reactions and increase in relative fluorescence
for the 1e5c RNA and 500c RNA samples.

| | Pore | 1e5c RNA | | 500c RNA | |
|---|---|---|---|---|---|
| | size | Cq | | Cq | |
| Membrane | (μm) | (min) | Max-dR | (min) | Max-dR |
| Cellulose acetate | 1.2 | 4.68 | 41044 | 6.76 | 33895 |
| mixed cellulose esters | 0.8 | 5.91 | 2925 | 9.86 | 1684 |
| mixed cellulose esters | 1 | 5.4 | 3078 | 8.54 | 1113 |
| polyacrylonitrile | 0.2 | 5.39 | 56963 | 4.69 | 70324 |
| polycarbonate track | 0.6 | 4.54 | 52262 | 6.39 | 51322 |
| etched | | | | | |
| polycarbonate track | 0.8 | 4.4 | 50215 | 6.25 | 52471 |
| etched | | | | | |
| polyethersulfone | 0.8 | 4.77 | 19098 | 7.33 | 8550 |
| polyester track etched | 0.8 | 4.91 | 7596 | 6.23 | 11874 |
| polyester track etched | 1 | 4.78 | 11061 | 7.07 | 14884 |
| polyvinylidene | 0.45 | 5.52 | 39677 | 4.43 | 39059 |

### Example 16

### Effect of Paramagnetic Particles and Membranes on RT-LAMP Amplification.

One possible iteration of a diagnostic device using specific target capture (STC) would have the lysis/hybridization solution removed from the magnetic particles by filtration instead of using a magnetic field, and the captured template amplified directly on the PMP retained on the membrane. This example demonstrates that RT-LAMP amplification can occur in the presence of PMP and a membrane, whether the RNA template is annealed on the beads or free in solution. In a first set of experiments, the specific target capture samples didn't contain any RNA template, and 1e5c RNA template was added later in the RT-LAMP reaction: in this case, the template in the amplification was not attached to the beads. In a second set of experiments, the specific target capture samples contained 5e5c RNA; in this case the RNA template was annealed to the beads at the beginning of the RT-LAMP reaction. Size exclusion devices were made as described in Example 14, using the following membranes: CA with 1.2 μm pores, MCE with 1.0 μm pores, PAN with 0.2 μm pores, PCTE with 0.8 μm pores, PES with 0.8 μm pores, PETE with 1 μm pores and PVDF with 0.45 μm pores. The samples comprised 100 μl of simulated nasal matrix (described in Example 6) and either SARS-COV-2 N-gene RNA or water. The samples were mixed with 504 μl of a lysis/hybridization buffer (120 mM Tris buffer pH 7.5, 12 mM EDTA, 48 mM ammonium sulfate, 120 mM lithium chloride, 0.12% SDS), 100 μg of Oligo-dT PMP and 2 pmoles each of 2 target-specific capture probes

CapN_358R:
(SEQ ID NO: 80)
CAGCTTCTGGCCCAGTTCCTAGGTAGTAAAAAAAAAAAAAAAAAAAAAA
AAAAAAAA
and
CapN_886R:
(SEQ ID NO: 81)
TTTCCTTGTCTGATTAGTTCCTGGTCCCAAAAAAAAAAAAAAAAAAAAA
AAAAAAAAA

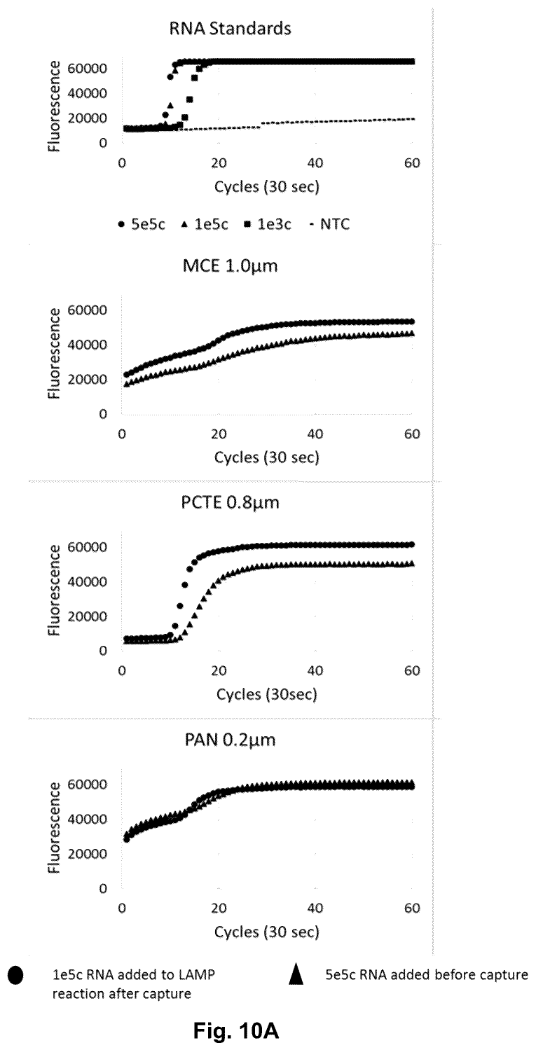

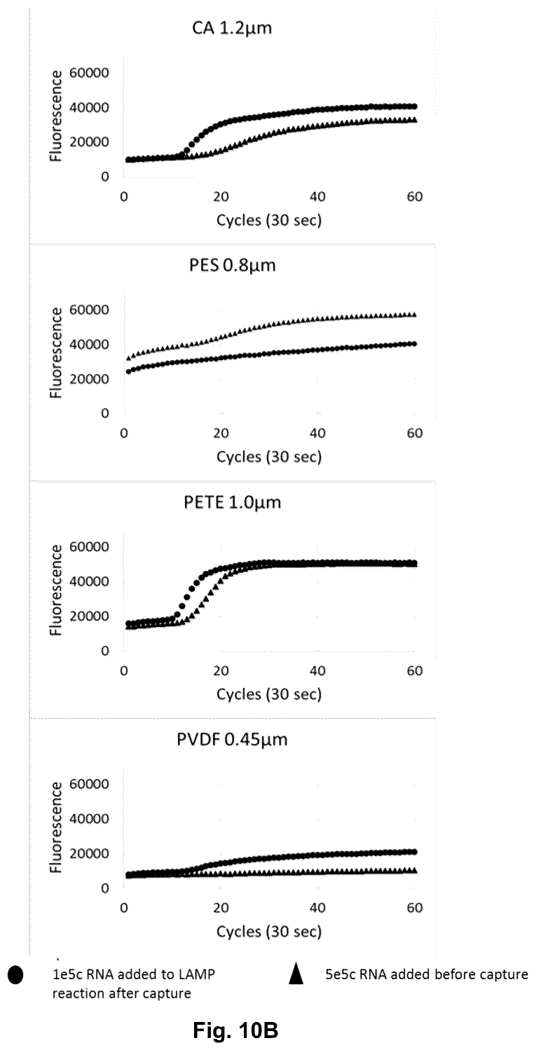

The samples were mixed by inverting the container and incubated for 10 minutes at 95° C. for the denaturation step, then 10 minutes at 60° C. for the hybridization step and then 10 minutes at room temperature for the immobilization step. The samples were then passed through the size exclusion device described in FIG. 8A. After the sample had passed through 600 μl wash buffer was added to the blue tip sample reservoir and allowed to flow through. The membranes with the PMPs were then carefully placed in PCR tubes so that they lined the outside of the tubes with the beads facing inside. 95 μl of RT-LAMP master mix and 5 μl of template (first set of experiments) or dH₂O (second set of experiments) were added to the PCR tubes. The primers used are listed in Table 16 and the RT-LAMP master mix composition was 1× iB5 buffer (Optigene, UK), 3 mM magnesium sulfate, 1 μM Syto-9, 1M Betaine, 32U GspM3.0 (Optigene, UK) and 2U AMV (Promega). The reactions were incubated for 60 minutes at 65° C. in a MxPro3005P thermocycler (Stratagene) with a read every 30 seconds. The results are shown in Table 18 and FIGS. 10A and 10B. The devices with the PCTE and PETE membranes gave the best results, with fast amplification, good signal intensity and stable baseline fluorescence. The CA membrane also performed relatively well. The PAN, PES and MCE membranes resulting in sloping baselines making the curves difficult to interpret. The PVDF sample with annealed RNA failed to amplify.

TABLE 18
Membrane used, time to amplification (Cq) of the RT-LAMP reactions
and increase in relative fluorescence for the samples.

| | Pore | 1e5c RNA in LAMP | | 5e5c RNA in STC | |
|---|---|---|---|---|---|
| | size | Cq | Fluorescence | Cq | Fluorescence |
| membrane | (μm) | (min) | increase | (min) | increase |
| Cellulose acetate | 1.2 | 5.9 | 23203 | 8.91 | 16581 |
| mixed cellulose esters | 1 | 10.21 | 3491 | 9.73 | 3455 |
| polyacrylonitrile | 0.2 | 6.41 | 9348 | 8.45 | 5137 |
| polycarbonate track | 0.8 | 4.82 | 50261 | 5.8 | 42267 |
| etched | | | | | |
| polyethersulfone | 0.8 | N/A | | 9.96 | 4462 |
| polyester track etched | 1 | 5.17 | 27836 | 6.36 | 29748 |
| polyvinylidene | 0.45 | 7.71 | 6214 | N/A | |

### Example 17

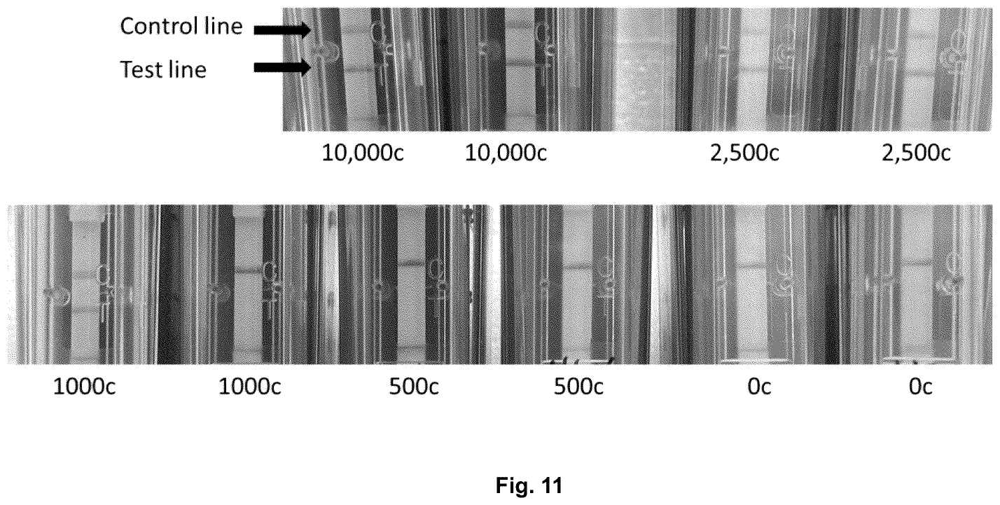

This example illustrates the detection of heat-inactivated SARS-COV-2 virus from a simulated nasal swab sample using the size exclusion device and detection with a lateral flow strip. Size exclusion devices were made as described in Example 14, using a polyester track etched membrane with a pore size of 1 μm. The samples comprised 10,000c, 2500c, 1000c, 500c or 0 c of heat-inactivated SARS-COV-2 virus (ATCC) in 25 μl Simulated Nasal Matrix (described in Example 6) and 75 μl 10×PBS. Each concentration was run in duplicates. The samples were mixed with 504 μl of a lysis/hybridization buffer (120 mM Tris buffer pH 7.5, 12 mM EDTA, 48 mM ammonium sulfate, 120 mM lithium chloride, 0.12% SDS), 100 μg of Oligo-dT PMP and 2 pmoles each of 2 target-specific capture probes listed in Table 19. The samples were mixed by inverting the container and incubated for 10 minutes at 95° C. for the denaturation step, then 30 minutes at 60° C. for the hybridization step and then 10 minutes at room temperature for the immobilization step. The samples were then passed through the size exclusion device described in FIG. 8A. After the sample had passed through 600 μl wash buffer was added to the blue tip sample reservoir and allowed to flow through. The membranes with the PMPs were then carefully placed in PCR tubes so that they lined the outside of the tubes with the beads facing inside. The membrane was then submerged with 100 μl RT-LAMP master mix composed of 1× iB5 buffer (Optigene, UK), 3 mM magnesium sulfate, 1 μM Syto-9, 1 M Betaine, 32 U GspM3.0 (Optigene, UK) and 2 U AMV (Promega). The primers used are listed in Table 19. The reactions were run for 30 minutes at 65° C. on a heat block. Each tube was then placed in an EasyNAT nucleic acid detection device (Ustar, China), which is a lateral flow strip encased in a contamination-proof plastic device. Amplification products with FAM and Biotin labels show as a red line on the test line of the strip. The results are shown in FIG. 11. The test accurately detected 2/2 samples with 10,000c or 2,500c virus, and ½ at 1,000 c virus. No signal was detected for samples containing 500c or 0 c of the virus.

TABLE 19
Target-specific capture probes used in Example 17.

| | | Final |
|---|---|---|
| Name | Sequence | (μM) |
| Cv19-N2-F3 | ACACAAGCTTTCGGCAGAC (SEQ ID NO: 82) | 0.2 |
| Cv19-N2-B3 | CCAATTTGATGGCACCTGTG (SEQ ID NO: 83) | 0.2 |
| Bio-Cv19-N2- | /5Biosg/TGTGCAATTTGCGGCCAATGTTTGGTCCAGA | 1.4 |
| FIP1 | ACAAACCCAAG (SEQ ID NO: 84) | |
| FAM-Cv19- | /56-FAM/ | 1.4 |
| N2-BIP | CCAGCGCTTCAGCGTTCTTCGTAGGTCAACCACGTT | |
| | CCCG (SEQ ID NO: 85) | |
| Cv19-N2-LF1 | CTGATTAGTTCCTGGTCCCCA (SEQ ID NO: 86) | 0.7 |
| Cv19-N2-LB1 | AATGTCGCGCATTGGCATGG (SEQ ID NO: 87) | 0.7 |
| Cap_N_358R | CAGCTTCTGGCCCAGTTCCTAGGTAGTAAAAAAAA | 1 pmole |
| | AAAAAAAAAAAAAAAAAAAAAA (SEQ ID NO: 88) | |
| Cap_N_886R | TTTCCTTGTCTGATTAGTTCCTGGTCCCAAAAAAAA | 1 pmole |
| | AAAAAAAAAAAAAAAAAAAAAA (SEQ ID NO: 89) | |

### Example 18

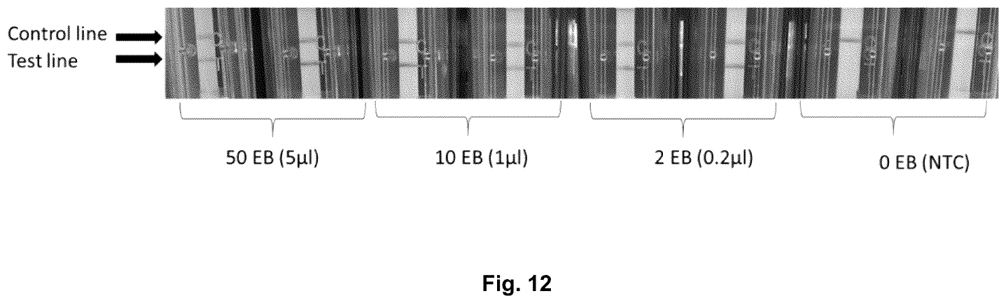

This example illustrates the detection of inactivated Chlamydia trachomatis (CT) cells (Acrometrix CT/NG control, ThermoFisher) from simulated vaginal swab samples after specific target capture using size exclusion devices and detection with lateral flow strips. Size exclusion devices were made as described in Example 14, using a polyethersulfone membrane with a pore size of 0.8 μm. Samples comprised 100 μl simulated vaginal fluid (described in Example 3) with 5 μl, 1 μl, 0.2 μl or 0 μl Acrometrix CT/NG control, with 4 replicates per concentration: each μl Acrometrix control contains 10 CT elementary bodies and 100 NG cells. The samples were mixed with 500 μl of a lysis/hybridization buffer (120 mM Tris buffer pH 7.5, 12 mM EDTA, 48 mM ammonium sulfate, 120 mM lithium chloride, 0.12% SDS), 100 μg of Oligo-dT PMP and 16 pmoles the capture probes mixture listed in Table 1. The samples were mixed by inverting the container and incubated for 10 minutes at 95° C. for the denaturation step, then 10 minutes at 60° C. for the hybridization step and then 10 minutes at room temperature for the immobilization step. The samples were then passed through the size exclusion device described in FIG. 8A. After the sample had passed through 600 μl wash buffer was added to the blue tip sample reservoir and allowed to flow through. The membranes with the PMPs were then carefully placed in PCR tubes so that they lined the outside of the tubes with the beads facing inside. Two replicates of each condition were used for lateral flow detection and two were used for real-time amplification. For lateral flow strip detection the membranes were submerged with 100 μl RT-LAMP master mix composed of 1× iB4 buffer (Optigene, UK), 3 mM magnesium sulfate, 0.4 mM each dNTP, 1M Betaine, 32 U GspF and 1× LAMP primers with FAM and Biotin labels. The LAMP primer sequences are listed in Table 20. The reactions were run for 30 minutes at 65° C. on a heat block. Each tube was then placed in an EasyNAT nucleic acid detection device (Ustar, China), which is a lateral flow strip encased in a contamination-proof plastic device. Amplification products with FAM and Biotin labels show as a red line on the test line of the strip. The results are shown in FIG. 12. For real-time detection, the membranes were submerged with 100 μl RT-LAMP master mix composed of 1× iB4 buffer (Optigene, UK), 3 mM magnesium sulfate, 0.4 mM each dNTP, 1 M Betaine, 1 μM Syto-9, 32 U GspF and 1× LAMP primers (listed in Table 1). The reactions were run for 30 minutes at 65° C. followed by a melt curve analysis in a MxPro3005P thermocycler (Stratagene). The results are shown in Table 21. We were able to detect as little as 2 CT elementary bodies per sample, using either the lateral flow strip detection or real-time detection.

TABLE 20
RT-LAMP primers used in Example 18.

| | | Final |
|---|---|---|
| | | concentration |
| LAMP primers | Sequence | (μM) |
| CT-LAMP4-F3 | CTGCTTGAGAGAACGTGCG (SEQ ID NO: 90) | 0.2 |
| CT-LAMP4-B3 | CTC TGA AGT CTT AAG CTT GGA GAT (SEQ ID | 0.2 |
| | NO: 91) | |
| CT-LAMP4-FIP | /5BioSg/TCGGTCAACGAAGAGGTTTTGCGATTT | 1.4 |
| | GCCTTAACCCCACC (SEQ ID NO: 92) | |
| CT-LAMP4-BIP | /56- | 1.4 |
| | FAM/AATCCTCTTTTCTGTCTGACGGTCAGATTT | |
| | GTTTCCAACAAGCTACC (SEQ ID NO: 93) | |
| CT-LAMP4-LoF | TTT TGT CTT CGT AAC TCG CT (SEQ ID NO: 94) | 0.7 |
| CT-LAMP4-LoB | TCT TAA GCT GGG AGA AAG AAA T (SEQ ID | 0.7 |
| | NO: 95) | |

TABLE 21
Real-time detection of Chlamydia trachomatis cells isolated
using a size exclusion device and amplified using LAMP. The
time to amplification (Cq), the intensity of the fluorescent
signal (Final RFU) and the melt temperature (Tm) are shown.

| Standards | SQ | Cq(min) | Final RFU | Tm(° C.) |
|---|---|---|---|---|
| CT gBlock | 5.00E+02 | 8.02 | 38001 | 83.7 |
| | 1.00E+02 | 8.45 | 35142 | 83.7 |
| | NTC | No Ct | | 57.0 |

| Input | | | | Final | | | | |
|---|---|---|---|---|---|---|---|---|
| (EBs) | | Samples | Cq(min) | RFU | Tm | AvgCq | STDEV | pos# |
| 50 | EB | A1 | 11.79 | 6344 | 83.7 | 12.13 | 0.48 | 2/2 |
| | | A2 | 12.46 | 7747 | 83.7 | | | |
| 10 | EB | B1 | 13.17 | 16266 | 83.7 | 15.79 | 3.71 | 2/2 |
| | | B2 | 18.41 | 4294 | 83.7 | | | |
| 2 | EB | C1 | 15.38 | 6391 | 83.7 | 15.70 | 0.46 | 2/2 |
| | | C2 | 16.03 | 12276 | 83.7 | | | |
| 0 | EB | D1 | No Ct | | 60.6 | No Ct | No Ct | 0/2 |
| | | D2 | No Ct | | 79.1 | | | |

### Example 19

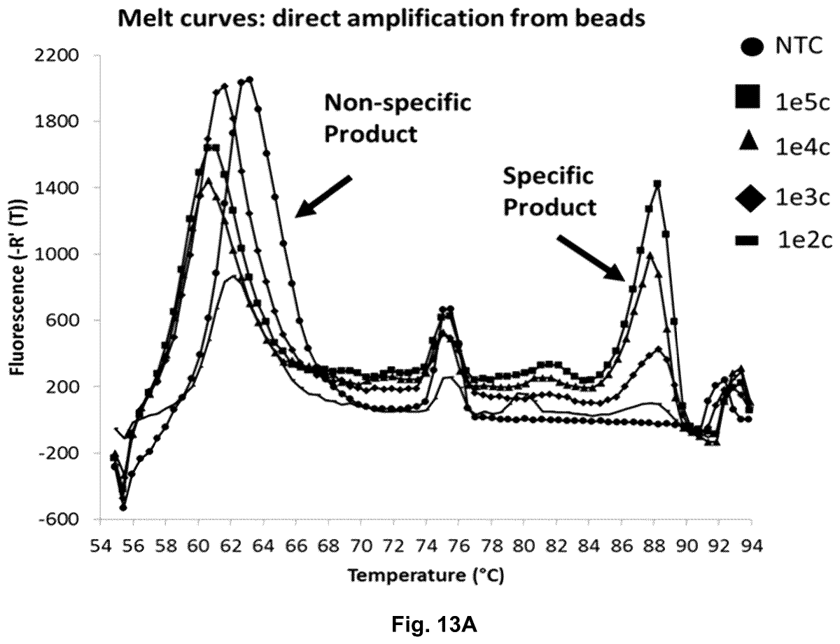

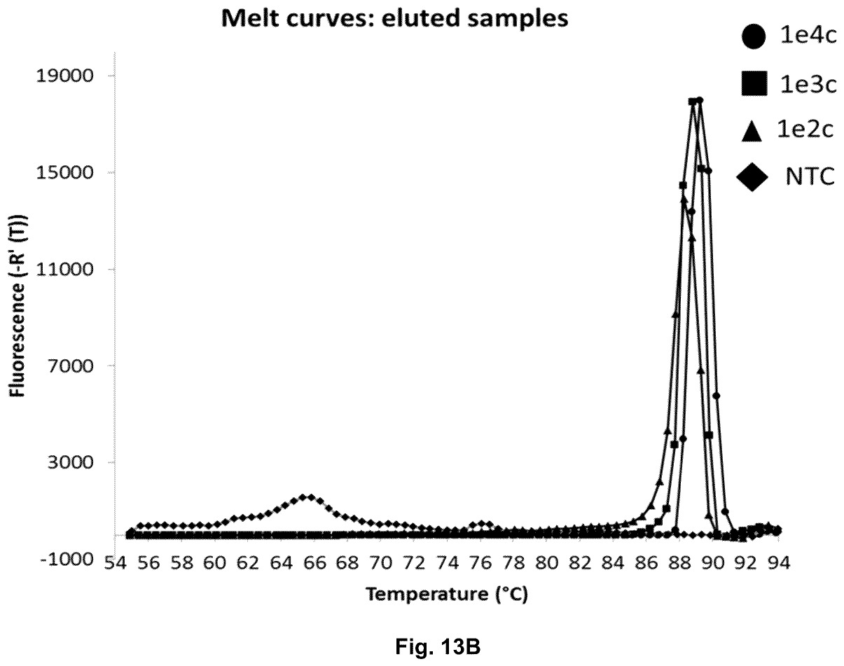

This example demonstrates the isolation of SARS-COV-2 RNA with a rapid specific target amplification protocol using a magnetic field followed by RT-LAMP amplification directly from the magnetic beads. The samples comprised 1e5c, 1e4c, 1e3c, 1e2c or 0 c of Twist SARS-COV-2 synthetic RNA control (Twist biosciences, #102019) in 100 μl transport media (10 mM HEPES pH 7.3, 3% LiDS, 1.5 mM EDTA, 1.5 mM EGTA). Six replicates were prepared for each concentration: in 4 of the samples the RNA was amplified directly from the magnetic particles whereas in the remaining two the RNA was eluted from the beads before amplification. The samples were mixed with 100 μl of a solution containing 100 mM HEPES pH 7.3, 7% LiDS, 800 mM LiCl, 1.5 mM EDTA, 1.5 mM EGTA and 64 pmoles each of the Cap probes N_358R and N_886R (Table 19). 100 μg washed Oligo-dT PMPs was added to each sample and the samples were incubated for 5 minutes at 95° C., 5 minutes at 60° C. and 4 minutes at 25° C. The containers were then placed in a magnetic field for 1 minute and the solution was removed. The PMP were washed with 200 μl wash buffer (10 mM Tris pH 7.5, 1.5 mM EDTA, 1.5 mM EGTA, 1% Triton-X100, 100 mM NaCl and 5 mM MgCl₂). The solution was carefully removed. For the direct amplification, the beads were re-suspended directly in 50 μl RT-LAMP master mix. For the eluted samples, the beads were resuspended in 20 μl elution buffer (10 mM Tris pH 7.5), incubated for 2 minutes at 60° C., placed back in the magnetic field for 1 minute and the eluates were carefully taken out and placed in the tubes for the RT-LAMP reactions. The RT-LAMP reactions composition was of 1× iB5 buffer (Optigene, UK), 3 mM magnesium sulfate, 1 μM Syto-9, 1M Betaine, 16 U GspM3.0 (Optigene, UK) and 1 U AMV (Promega) for a total volume of 50 μl. The LAMP reactions were run for 30 minutes at 65° C. followed by a melt curve analysis. The results show that direct RT-LAMP amplification from Oligo-dT PMPs with captured RNA is possible but that the reactions are both slower and less sensitive than reactions from the eluted samples (Table 22). The melting curves of the reactions also showed that direct amplification resulted in non-specific product with a low melting temperature in addition to the specific LAMP amplification product (FIGS. 13A and 13B).

TABLE 22
Average time to amplification (AvgCq) and number
of positives of direct amplification on the magnetic
particles compared to RT-LAMP on eluates.

| | RNA | AvgCq | | |
|---|---|---|---|---|
| Protocol | Input | (min) | STDEV | pos# |
| Direct | 1e5c | 13.09 | 5.17 | 4/4 |
| | 1e4c | 12.99 | 3.38 | 3/4 |
| | 1e3c | 17.61 | 0 | 2/4 |
| | 1e2c | 15.81 | 0 | 1/4 |
| | NTC | no amplification | | 0/4 |
| Eluted | 1e4c | 5.26 | 0.05 | 2/2 |
| | 1e3c | 6.25 | 0.06 | 2/2 |
| | 1e2c | 9.07 | 2.16 | 2/2 |
| | NTC | no amplification | | 0/2 |
| Standards | 1e5c | 4.33 | 0.11 | 2/2 |
| | 1e4c | 4.92 | 0.32 | 2/2 |
| | 1e3c | 5.68 | 0.24 | 2/2 |
| | 1e2c | 6.5 | 0.4 | 2/2 |

| | | |
|---|---|---|
| NTC | no amplification | 0/2 |

### Example 20 (Prophetic)

This example demonstrates that methods provided herein can be further expanded to other amplification methods, including but not limited to, signal amplification for the detection of nucleic acid sequence. A sample is processed according to the methods described above and DNA or RNA is isolated. The DNA or RNA can now be detected with an enzymatic reaction, for instance a detection method mediated by CRISPR-Cas enzymes, such as Cas12a or Cas13a as is known in the art.

1. A method of capturing and processing a target nucleic acid, the method comprising:
immobilizing a target nucleic acid on a solid substrate in contact with a first solution, wherein:
the immobilizing comprises a step of hybridizing the target nucleic acid to a capture oligomer configured to bind to the target nucleic acid to generate a target complex; and
the first solution comprises:
water;
salt; and
a first reagent comprising at least one of a detergent and a protease;
removing the first solution from the immobilized target complex; and
enzymatically processing the target nucleic acid.

2-63. (canceled)

64. The method of claim 1, further comprising combining a clinical sample and one or more of the water, the salt, and the first reagent to generate the first solution.

65. The method of claim 1, wherein the clinical sample comprises at least one of whole blood, serum, plasma, sputum, saliva, nasopharyngeal swab, stool, anal swab, vaginal swab, urine, dry blood spot, penile swab, urethral swab, and skin swab.

66. The method of claim 1, further comprising lysing cells and/or denaturing the target nucleic acid in the first solution, comprising heating the first solution to a first temperature and incubating the first solution at the first temperature for a time from 30 seconds to 3 minutes, wherein the first temperature is from 60° C. to 110° C.

67. The method of claim 66, wherein the solid substrate is in contact with the first solution during the hybridizing and the lysing and/or denaturing.

68. The method of claim 1, wherein the hybridizing comprises cooling the first solution to a second temperature and incubating the first solution at the second temperature for a time from 30 seconds to 3 minutes, wherein the second temperature is from 30° C. to 75° C.

69. The method of claim 1, wherein the solid substrate comprises a bead.

70. The method of claim 1, wherein:
the capture oligomer comprises a first binding moiety of a specific binding pair, wherein the first binding moiety is capable of binding to a second binding moiety of the specific binding pair and the second binding moiety of the specific binding pair is bound to or is capable of binding to the solid substrate; and
the immobilizing comprises binding the first binding moiety to the second binding moiety, comprising cooling the first solution to a third temperature and incubating the first solution at the third temperature for a time from 30 seconds to 3 minutes, wherein the third temperature is from 10° C. to 50° C.

71. The method of claim 70, wherein the specific binding pair comprises streptavidin and biotin; hybridizable nucleic acid sequences; an antibody and an antigen of the antibody; a G-quadruplex structure and a G-quadruplex-binding protein; an aptamer and an aptamer target; and an ion/anion binding pair.

72. The method of claim 70, wherein the specific binding pair comprises hybridizable nucleic acid sequences.

73. The method of claim 1, wherein:
the solid substrate comprises a magnetic substrate; and
the removing the first solution comprises:
immobilizing the magnetic substrate with a magnetic field; and
separating the first solution from the immobilized magnetic substrate.

74. The method of claim 1, wherein the first solution comprises the protease, the protease comprises proteinase K, and the proteinase K is present in the first solution in an amount of 3 to 300 Units.

75. The method of claim 1, wherein the salt is present in the first solution in an amount effective to result in a molar ionic strength equivalent to 50 mM to 1 M NaCl.

76. The method of claim 1, wherein the immobilized target complex is not washed or is washed only once after the removing the first solution from the immobilized target complex and prior to the enzymatically processing the target nucleic acid.

77. The method of claim 1, wherein the immobilized target complex is not rinsed or is rinsed three or fewer times after the removing the first solution from the immobilized target complex and prior to the enzymatically processing the target nucleic acid.

78. The method of claim 1, wherein the target nucleic acid is enzymatically processed as part of the immobilized target complex without eluting the target nucleic acid from the capture oligomer.

79. The method of claim 1, comprising:
prior to the immobilizing, heating the first solution with the target nucleic acid therein to a temperature of 60° C. to 110° C. to generate a heated solution; and then
exposing the heated solution to a temperature of 20-25° C. for a time sufficient to hybridize the target nucleic acid to the capture oligomer and immobilize the target nucleic acid on the solid substrate.

80. The method of claim 1, wherein:
the method further comprises lysing cells and/or denaturing the target nucleic acid in the first solution, comprising heating the first solution to a first temperature and incubating the first solution at the first temperature for a time from 1 minute to 3 minutes, wherein the first temperature is from 60° C. to 110° C.;
the hybridizing comprises cooling the first solution to a second temperature and incubating the first solution at the second temperature for a time from 1 to 3 minutes, wherein the second temperature is from 30° C. to 75° C.;
the capture oligomer comprises a first binding moiety of a specific binding pair, wherein the first binding moiety is capable of binding to a second binding moiety of the specific binding pair, the specific binding pair comprises hybridizable nucleic acid sequences, and the second binding moiety of the specific binding pair is bound to the solid substrate;
the immobilizing comprises binding the first binding moiety to the second binding moiety, comprising cooling the first solution to a third temperature and incubating the first solution at the third temperature for a time from 1 minute to 3 minutes, wherein the third temperature is from 10° C. to 50° C.;
the solid substrate is in contact with the first solution during the hybridizing and the lysing and/or denaturing;
the first solution comprises the protease, the protease comprises proteinase K, and the proteinase K is present in the first solution in an amount of 3 to 300 Units;
the salt is present in the first solution in an amount effective to result in a molar ionic strength equivalent to 50 mM to 1 M NaCl;
the immobilized target complex is not washed or is washed only once after the removing the first solution from the immobilized target complex and prior to the enzymatically processing the target nucleic acid; and
the immobilized target complex is not rinsed or is rinsed three or fewer times after the removing the first solution from the immobilized target complex and prior to the enzymatically processing the target nucleic acid.

81. The method of claim 80, wherein the first solution comprises the detergent and the detergent comprises at least one of a dodecyl sulfate salt and t-octylphenoxypolyethoxyethanol in amount from 0.05% w/v to 5% w/v.

82. The method of claim 80, wherein the target nucleic acid is enzymatically processed as part of the immobilized target complex without eluting the target nucleic acid from the capture oligomer.


SEQUENCE LISTING (98 sequences)

SEQ ID NO: 1 - DNA, 57 bp, Artificial Sequence
  Capture oligomer CT_CP-cap840R
cttcgagcaaccgctgtgacggagtacaaaaaaaaaaaaaaaaaaaaaaaaaaaaaa

SEQ ID NO: 2 - DNA, 57 bp, Artificial Sequence
  Capture oligomer CT_CP-cap881R
ctcagactccgacataatgaaggtcacaaaaaaaaaaaaaaaaaaaaaaaaaaaaaa

SEQ ID NO: 3 - DNA, 57 bp, Artificial Sequence
  Capture oligomer CT_CP-cap560F
cctccttggagcattgtctgggcgatcaaaaaaaaaaaaaaaaaaaaaaaaaaaaaa

SEQ ID NO: 4 - DNA, 56 bp, Artificial Sequence
  Capture oligomer CT_CP-cap505F
gctcttttaggaaggatgctgtttgcaaaaaaaaaaaaaaaaaaaaaaaaaaaaaa

SEQ ID NO: 5 - DNA, 58 bp, Artificial Sequence
  Capture oligomer NG_ISNgo2-328R
accggcaatactgcgctatcagttttgcaaaaaaaaaaaaaaaaaaaaaaaaaaaaaa

SEQ ID NO: 6 - DNA, 57 bp, Artificial Sequence
  Capture oligomer NG_ISNgo2-613F
tgaccgcagccgtattgtttcatcatcaaaaaaaaaaaaaaaaaaaaaaaaaaaaaa

SEQ ID NO: 7 - DNA, 19 bp, Artificial Sequence
  LAMP primer CT-LAMP4-F3
ctgcttgagagaacgtgcg

SEQ ID NO: 8 - DNA, 24 bp, Artificial Sequence
  LAMP primer CT-LAMP4-B3
ctctgaagtcttaagcttggagat

SEQ ID NO: 9 - DNA, 41 bp, Artificial Sequence
  LAMP primer CT-LAMP4-FIP
tcggtcaacgaagaggttttgcgatttgccttaaccccacc

SEQ ID NO: 10 - DNA, 47 bp, Artificial Sequence
  LAMP primer CT-LAMP4-BIP
aatcctcttttctgtctgacggtcagatttgtttccaacaagctacc

SEQ ID NO: 11 - DNA, 20 bp, Artificial Sequence
  LAMP primer CT-LAMP4-LF
ttttgtcttcgtaactcgct

SEQ ID NO: 12 - DNA, 22 bp, Artificial Sequence
  LAMP primer CT-LAMP4-LB
tcttaagctgggagaaagaaat

SEQ ID NO: 13 - DNA, 27 bp, Artificial Sequence
  Capture oligomer capN_358R
cagcttctggcccagttcctaggtagt

SEQ ID NO: 14 - DNA, 33 bp, Artificial Sequence
  Capture oligomer capN_886R
aaaaatttccttgtctgattagttcctggtccc

SEQ ID NO: 15 - DNA, 19 bp, Artificial Sequence
  LAMP primer Cv19-N2-F3
acacaagctttcggcagac

SEQ ID NO: 16 - DNA, 20 bp, Artificial Sequence
  LAMP primer Cv19-N2-B3
ccaatttgatggcacctgtg

SEQ ID NO: 17 - DNA, 42 bp, Artificial Sequence
  LAMP primer Cv19-N2-FIP1
tgtgcaatttgcggccaatgtttggtccagaacaaacccaag

SEQ ID NO: 18 - DNA, 40 bp, Artificial Sequence
  LAMP primer Cv19-N2-BIP
ccagcgcttcagcgttcttcgtaggtcaaccacgttcccg

SEQ ID NO: 19 - DNA, 21 bp, Artificial Sequence
  LAMP primer Cv19-N2-LF1
ctgattagttcctggtcccca

SEQ ID NO: 20 - DNA, 20 bp, Artificial Sequence
  LAMP primer Cv19-N2-LB1
aatgtcgcgcattggcatgg

SEQ ID NO: 21 - DNA, 17 bp, Artificial Sequence
  qPCR primer N_653F
ctcttgctttgctgctg

SEQ ID NO: 22 - DNA, 18 bp, Artificial Sequence
  qPCR primer N_852R
tccttgggtttgttctgg

SEQ ID NO: 23 - DNA, 20 bp, Artificial Sequence
  qPCR primer F370
acgagcaatacaggctttca

SEQ ID NO: 24 - DNA, 19 bp, Artificial Sequence
  qPCR primer R479
ttggccgcttcttcatctt

SEQ ID NO: 25 - DNA, 33 bp, Artificial Sequence
  Capture oligomer nsp1_88R
aaaaatcacgtacgagcacgtcgcgaacctgta

SEQ ID NO: 26 - DNA, 32 bp, Artificial Sequence
  Capture oligomer nsp1_350R
aaaaatgccactggtatttcgcccacatgagg

SEQ ID NO: 27 - DNA, 21 bp, Artificial Sequence
  qPCR primer 145F
ggcacttgtggcttagtagaa

SEQ ID NO: 28 - DNA, 21 bp, Artificial Sequence
  qPCR primer 270R
tgctaccagctcaaccataac

SEQ ID NO: 29 - DNA, 32 bp, Artificial Sequence
  Capture oligomer TBkatG_capF3
aaaaaatgggtcttaccgaaagtgtgaccgcc

SEQ ID NO: 30 - DNA, 32 bp, Artificial Sequence
  Capture oligomer TBkatG_capR2
aaaaagcatcgaggtcgtatggacgaacaccc

SEQ ID NO: 31 - DNA, 18 bp, Artificial Sequence
  qPCR primer TB-katG-F2
cgtccttggcggtgtatt

SEQ ID NO: 32 - DNA, 21 bp, Artificial Sequence
  qPCR primer TB-KatG-R2
ggacaacagtttcctcgagat

SEQ ID NO: 33 - DNA, 19 bp, Artificial Sequence
  qPCR primer TB-katG-1645F
tgtgccgccatagagaaag

SEQ ID NO: 34 - DNA, 19 bp, Artificial Sequence
  qPCR primer TB-katG-1750F
aggattccacgtcggtttg

SEQ ID NO: 35 - DNA, 19 bp, Artificial Sequence
  qPCR primer TB-katG-2116F
gcgcttgtcgaggtctatg

SEQ ID NO: 36 - DNA, 20 bp, Artificial Sequence
  qPCR primer TB-katG-2210F
aacctgtcgaggttcatcac

SEQ ID NO: 37 - DNA, 19 bp, Artificial Sequence
  qPCR primer TB-katG-3333F
accgaagacggcaattgat

SEQ ID NO: 38 - DNA, 21 bp, Artificial Sequence
  qPCR primer TB-katG-3453R
gttacccatccgcttccaata

SEQ ID NO: 39 - DNA, 20 bp, Artificial Sequence
  qPCR primer TB-katG-4231F
tgatcaactgtgctggtctg

SEQ ID NO: 40 - DNA, 22 bp, Artificial Sequence
  qPCR primer TB-katG-4337R
cagttgctctagacctggattg

SEQ ID NO: 41 - DNA, 20 bp, Artificial Sequence
  qPCR primer TB-katG-5268F
tggcaagcagtgtgagtaag

SEQ ID NO: 42 - DNA, 19 bp, Artificial Sequence
  qPCR primer TB-katG-5353R
cgcacactagagccacaaa

SEQ ID NO: 43 - DNA, 32 bp, Artificial Sequence
  Capture oligomer TBahpC_capR
aaaaagcgagcggtggtcggaagccggtctcg

SEQ ID NO: 44 - DNA, 32 bp, Artificial Sequence
  Capture oligomer TBahpC_capF
aaaaagtggcgggtggtgttcttttggccgaa

SEQ ID NO: 45 - DNA, 33 bp, Artificial Sequence
  Capture oligomer TBembB_capR
aaaaagcccgacgggtccttgagcccgacgaag

SEQ ID NO: 46 - DNA, 32 bp, Artificial Sequence
  Capture oligomer TBembB_capF
aaaaactcacaccggcggcgctggccgtcgtt

SEQ ID NO: 47 - DNA, 33 bp, Artificial Sequence
  capture oligomer TBinhA_capR
aaaaagcgggtgtattgacgccctacgaattgc

SEQ ID NO: 48 - DNA, 32 bp, Artificial Sequence
  Capture oligomer TBinhA_capF
aaaaagcacagcggctggctgccgacggccac

SEQ ID NO: 49 - DNA, 32 bp, Artificial Sequence; identical to SEQ ID NO: 29
  Capture oligomer TBkatG_capF3

SEQ ID NO: 50 - DNA, 32 bp, Artificial Sequence; identical to SEQ ID NO: 30
  Capture oligomer TBkatG_capR2

SEQ ID NO: 51 - DNA, 31 bp, Artificial Sequence
  Capture oligomer TBpncA_CapR1
aaaaatcccagtctggacacgtcggcaatcg

SEQ ID NO: 52 - DNA, 32 bp, Artificial Sequence
  Capture oligomer TBpncA_CapF1
aaaaaccttggttgccacgacgtgatggtagt

SEQ ID NO: 53 - DNA, 32 bp, Artificial Sequence
  Capture oligomer TBrpoB_capF4
aaaaagcgatcacaccgcagacgttgatcaac

SEQ ID NO: 54 - DNA, 32 bp, Artificial Sequence
  Capture oligomer TBrpoB_capR3
aaaaaaagtggtcgatgtcgtcggtttccacc

SEQ ID NO: 55 - DNA, 28 bp, Artificial Sequence
  Capture oligomer TB-embBpr-CapR6
aaaaacggttaagggcggtgaggatgcg

SEQ ID NO: 56 - DNA, 28 bp, Artificial Sequence
  Capture oligomer TB-embBpr-CapF6
aaaaaatcgcacgcctagcagccgtcgt

SEQ ID NO: 57 - DNA, 20 bp, Artificial Sequence
  PCR primer 19-111_TB-rpoB-dis2_F
gacgagtgcaaagacaagga

SEQ ID NO: 58 - DNA, 20 bp, Artificial Sequence
  PCR primer 19-113_TB-rpoB-dis3_F
tcgtctgaggtggactacat

SEQ ID NO: 59 - DNA, 20 bp, Artificial Sequence
  PCR primer 19-114_TB-rpoB-dis3_R
gctccaggaagggaatcatc

SEQ ID NO: 60 - DNA, 20 bp, Artificial Sequence
  PCR primer 19-115_TB-rpoB-dis4_F
cgtgtgtatgtggctcagaa

SEQ ID NO: 61 - DNA, 18 bp, Artificial Sequence
  PCR primer 19-116_TB-rpoB-dis4_R
ggaacggcatgtcctcaa

SEQ ID NO: 62 - DNA, 20 bp, Artificial Sequence
  PCR primer 19-117_TB-rpoB-dis5_F
tgctcgacgtcaacttcttc

SEQ ID NO: 63 - DNA, 19 bp, Artificial Sequence
  PCR primer 19-118_TB-rpoB-dis5_R
gttgatcgtctccggcttt

SEQ ID NO: 64 - DNA, 19 bp, Artificial Sequence
  PCR primer 19-119_TB-rpoB_dis6_F
agatcgaggccgagctatt

SEQ ID NO: 65 - DNA, 21 bp, Artificial Sequence
  PCR primer 19-120_TB-rpoB-dis6_R
tgcttgttgacgaacggataa

SEQ ID NO: 66 - DNA, 20 bp, Artificial Sequence
  PCR primer 19-121_TB-rpoB-dis7_F
atcccgtcgtatgaggatca

SEQ ID NO: 67 - DNA, 19 bp, Artificial Sequence
  PCR primer 19-122_TB-rpoB-dis7_R
tagtcgctgtagccgtagt

SEQ ID NO: 68 - DNA, 18 bp, Artificial Sequence
  qPCR primer 19-075_TB-rpoB-F3
cacaccgcagacgttgat

SEQ ID NO: 69 - DNA, 22 bp, Artificial Sequence
  qPCR primer 19-076_TB-rpoB-R3
ggttgttctggtccatgaattg

SEQ ID NO: 70 - DNA, 30 bp, Artificial Sequence
  Capture oligomer EC-uidA-754R
gttcatagagataaccttcacccggttgcc

SEQ ID NO: 71 - DNA, 28 bp, Artificial Sequence
  Capture oligomer EC-uidA-887F
ttggtcgtcatgaagatgcggatttgcg

SEQ ID NO: 72 - DNA, 23 bp, Artificial Sequence
  qPCR primer EC_uidAF
ccaaaagccagacagagtgtgat

SEQ ID NO: 73 - DNA, 24 bp, Artificial Sequence
  qPCR primer EC_uidAR
agccagtaaagtagaacggtttgt

SEQ ID NO: 74 - DNA, 19 bp, Artificial Sequence
  LAMP primer Cv19N-F3_501F
gccaaaaggcttctacgca

SEQ ID NO: 75 - DNA, 20 bp, Artificial Sequence
  LAMP primer Cv19N-B3_740R
gtgacagtttggccttgttg

SEQ ID NO: 76 - DNA, 40 bp, Artificial Sequence
  LAMP primer Cv19N-FIP_539F
tcccctactgctgcctggaggtcaagcctcttctcgttcc

SEQ ID NO: 77 - DNA, 41 bp, Artificial Sequence
  LAMP primer Cv19N-BIP_720R
caatggcggtgatgctgctctttgttggcctttaccagaca

SEQ ID NO: 78 - DNA, 20 bp, Artificial Sequence
  LAMP primer Cv19N-LF_579R
actgttgcgactacgtgatg

SEQ ID NO: 79 - DNA, 20 bp, Artificial Sequence
  LAMP primer Cv19N-LB_677F
gattgaaccagcttgagagc

SEQ ID NO: 80 - DNA, 57 bp, Artificial Sequence
  Capture probe CapN_358R
cagcttctggcccagttcctaggtagtaaaaaaaaaaaaaaaaaaaaaaaaaaaaaa

SEQ ID NO: 81 - DNA, 58 bp, Artificial Sequence
  Capture probe CapN_886R
tttccttgtctgattagttcctggtcccaaaaaaaaaaaaaaaaaaaaaaaaaaaaaa

SEQ ID NO: 82 - DNA, 19 bp, Artificial Sequence
  Capture probe Cv19-N2-F3
acacaagctttcggcagac

SEQ ID NO: 83 - DNA, 20 bp, Artificial Sequence
  Capture probe Cv19-N2-B3
ccaatttgatggcacctgtg

SEQ ID NO: 84 - DNA, 42 bp, Artificial Sequence; identical to SEQ ID NO: 17
  Capture probe Bio-Cv19-N2-FIP1

SEQ ID NO: 85 - DNA, 40 bp, Artificial Sequence; identical to SEQ ID NO: 18
  Capture probe FAM-Cv19-N2-BIP

SEQ ID NO: 86 - DNA, 21 bp, Artificial Sequence
  Capture probe Cv19-N2-LF1
ctgattagttcctggtcccca

SEQ ID NO: 87 - DNA, 20 bp, Artificial Sequence
  Capture probe Cv19-N2-LB1
aatgtcgcgcattggcatgg

SEQ ID NO: 88 - DNA, 57 bp, Artificial Sequence; identical to SEQ ID NO: 80
  Capture probe Cap_N_358R

SEQ ID NO: 89 - DNA, 58 bp, Artificial Sequence; identical to SEQ ID NO: 81
  Capture probe Cap_N_886R

SEQ ID NO: 90 - DNA, 19 bp, Artificial Sequence
  LAMP primer CT-LAMP4-F3
ctgcttgagagaacgtgcg

SEQ ID NO: 91 - DNA, 24 bp, Artificial Sequence
  LAMP primer CT-LAMP4-B3
ctctgaagtcttaagcttggagat

SEQ ID NO: 92 - DNA, 41 bp, Artificial Sequence; identical to SEQ ID NO: 9
  LAMP primer CT-LAMP4-FIP

SEQ ID NO: 93 - DNA, 47 bp, Artificial Sequence; identical to SEQ ID NO: 10
  LAMP primer CT-LAMP4-BIP

SEQ ID NO: 94 - DNA, 20 bp, Artificial Sequence
  LAMP primer CT-LAMP4-LoF
ttttgtcttcgtaactcgct

SEQ ID NO: 95 - DNA, 22 bp, Artificial Sequence
  LAMP primer CT-LAMP4-LoB
tcttaagctgggagaaagaaat

SEQ ID NO: 96 - DNA, 14 bp, Artificial Sequence
  Oligo-dT
tttttttttttttt

SEQ ID NO: 97 - DNA, 15 bp, Artificial Sequence
  Modified Oligo-dT with 3′ inverted dT
  modified_base (15)..(15): 3′ inverted dT
ttttttttttttttt

SEQ ID NO: 98 - DNA, 15 bp, Artificial Sequence
  Modified Oligo-dT with L-DNA dT
  modified_base (15)..(15): L-DNA dT
ttttttttttttttt